(19) United States
(12) Patent Application Publication
(10) Pub. No.: US 2016/0354472 A1
(43) Pub. Date: Dec. 8, 2016

(54) USE OF HUMAN SERUM ALBUMIN TO DECREASE ANTIGENICITY OF THERAPEUTIC PROTEINS

(71) Applicant: Fahar Merchant, West Vancouver (CA)

(72) Inventor: Fahar Merchant, West Vancouver (CA)

(73) Assignee: PROTOX THERAPEUTICS CORP., LaJolla, CA (US)

(21) Appl. No.: 13/280,165

(22) Filed: Oct. 24, 2011

Related U.S. Application Data
(60) Provisional application No. 61/406,052, filed on Oct. 22, 2010.

(51) Int. Cl. A61K 47/42; A61K 38/16; A61K 38/20; A61K 47/02; A61K 47/18

(52) U.S. Cl. CPC A61K 47/42; A61K 47/02; A61K 47/183; A61K 38/2026; A61K 38/164

(57) ABSTRACT

Provided herein are therapeutic compositions that include albumin (such as human serum albumin) and therapeutic proteins, such as a modified proaerolysin protein or an apoptosis-modulating fusion protein, as well as kits that include such compositions in containers. Also provided are methods of using such compositions in decreasing the antigenicity of therapeutic proteins such as modified proaerolysin proteins or apoptosis-modulating fusion protein antibodies (for example as evidenced by a decrease in the production of neutralizing antibodies).

This application claims the benefit of priority of U.S. provisional application No. 61/406,052, filed Oct. 22, 2010, the disclosure of which is hereby incorporated by reference as if written herein in its entirety.

### FIELD

This application relates to compositions that include albumin and a therapeutic protein (such as a modified proaerolysin protein), and their use in decreasing the antigenicity of such proteins, such as those used in cancer therapy.

### BACKGROUND

The immunogenicity of therapeutic proteins is widely recognized as a potential complication of their use (see, for example, Schellekens, Clinical Therapeutics 24: 1720, 2002 and U.S. Pat. No. 7,579,316). In particular, administration of therapeutic proteins having non-human sequences to a human patient can lead to undesirable immune responses against the therapeutic protein. For example, therapeutic proteins may elicit some level of antibody response when administered to a subject, which in some cases can lead to potentially serious side effects. The production of antibodies that are capable of neutralizing the biological effect of a therapeutic protein is a concern if the therapeutic protein is to be administered repeatedly. Thus, methods of reducing undesirable immune responses to therapeutic proteins are needed.

### SUMMARY

It has been surprisingly discovered that addition of albumin, such as human serum albumin (HSA), to a composition that includes a therapeutic protein (such as a modified proaerolysin (PA) protein, for example SEQ ID NO: 4 or SEQ ID NO: 28, targeted cargo protein, or an apoptosis-modifying fusion protein that includes an inactive toxin domain and an apoptosis regulating domain), reduces the immunogenicity of the therapeutic protein. In contrast to prior methods, generation of a fusion protein is not necessary to decrease immunogenicity (e.g., an albumin-modified fusion protein, such as that described in U.S. Pat. No. 6,946,134). Instead, addition of albumin to a composition containing a therapeutic protein is sufficient. In particular examples, albumin is added in excess over the therapeutic protein, such as at least 50 times (for example at least 100 times, at least 500 times, or at least 1000 times) more albumin than the therapeutic protein (such as a modified PA protein, targeted cargo protein, or apoptosis-modifying fusion protein) on a mole:mole basis. It is shown herein that administration of such compositions to human subjects reduces the immunogenicity of the therapeutic protein (such as a modified PA protein, targeted cargo protein, or apoptosis-modifying fusion protein) as indicated by the presence of antibodies specific for the therapeutic protein detected in serum, for example by at least 10% (such as at least 20%, at least 50%, at least 75%, at least 80%, at least 90%, or at least 95%, such as 10% to 90%) as compared to compositions that did not include albumin.

Based on these observations, disclosed are compositions that include albumin (such as human serum albumin, HSA) and a therapeutic protein (such as a modified PA protein, for example, the sequence shown in SEQ ID NO: 4 or SEQ ID NO: 28, or a targeted cargo protein that includes an targeting moiety, such as an inactive toxin domain that can target the protein to a cell and cargo moiety that mediates an effect on the cell, such as an apoptosis regulating domain), wherein the albumin reduces the immunogenicity of the therapeutic protein. For example, the presence of a sufficient amount of albumin can reduce the immunogenicity of the therapeutic protein by at least 2-fold, for example at least 3-fold, at least 4-fold, at least 5-fold, or at least 10-fold as compared to compositions that do not include albumin. In one example, the composition does not include a botulinum toxin. In another example the composition does not include the circularly permutted IL-4-Pseudomonas exotoxin PRX321 shown in FIG. 6.

Exemplary modified PA proteins include those having a prostate-specific protease cleavage site that functionally replaces a native proaerolysin furin cleavage site (such as the furin site corresponding to amino acids 427-432 of SEQ ID NO: 2). In particular examples, the prostate-specific protease cleavage site includes a prostate-specific antigen (PSA) cleavage site, such as those shown in SEQ ID NOS: 5-15. In some examples, the modified proaerolysin further includes a modified binding domain (e.g., shown as amino acids 1-83 of SEQ ID NO: 2 or 4), such as a deletion of such a domain or replacement with a prostate-tissue specific binding domain. In some examples, the modified proaerolysin further includes a polyhistidine tag (such as a 6-His tag at the N- or C-terminus of the modified proaerolysin protein). Kits that include the disclosed compositions are also provided.

In particular examples, the apoptosis-modifying fusion protein includes an inactive toxin domain and an apoptosis regulating domain, wherein the inactive toxin domain targets the therapeutic protein to a cell. For example, such therapeutic fusion proteins can include an apoptosis-modifying fragment or variant thereof from the Bcl-2 protein family (such as Bcl-x_{L} or Bad) and a cell-binding, targeting domain derived from a bacterial toxin. Such therapeutic proteins can either regulate cell viability either positively (using anti-death Bcl-2 family members) or negatively (using pro-death members of the Bcl-2 family). The proteins can be target to specific subsets of cells permitting treatment and/or prevention of the cell-death related consequences of various diseases and injuries. Certain embodiments will also include a linker between these two domains. This linker can be at least 5 amino acids long, for example between 5 and 100 amino acids in length, and may for instance include the amino acid sequence shown in SEQ ID NO: 23. Appropriate linkers can be 6, 7, or 8 amino acids in length, and so forth, including linkers of about 10, 20, 30, 40 or 50 amino acids long. The apoptosis modifying fusion proteins can also include a third domain from one of the two original proteins, or from a third protein. This third domain may improve the fusion protein's ability to be integrated into or otherwise cross a cellular membrane of the target cell. An example of such a third domain is the translocation region (domain or sub-domain) of diphtheria toxin. Target cells for the fusion proteins disclosed herein include, but are not limited to, neurons, lymphocytes, stem cells, epithelial cells, cancer cells, neoplasm cells, and others, including other hyper-proliferative cells. The target cell chosen will depend on what disease or injury condition the fusion protein is intended to treat.

In one example, the apoptosis-modifying fusion protein includes essentially the entire Bcl-x_{L} protein as the apoptosis-modifying domain of the fusion protein, or variants or fragments thereof that maintain the ability to inhibit apoptosis in a target cell to which the protein is exposed. Examples of such proteins are fusion proteins made of the Bcl-x_{L} protein, functionally linked to the diphtheria toxin receptor binding domain through a peptide linker of about six amino acids. One such protein is Bcl-x_{L}-DTR, which consists of Bcl-x_{L} and DTR, without the translocation domain of diphtheria toxin. The nucleotide sequence of this fusion protein is shown in SEQ ID NO: 18, and the corresponding amino acid sequence in SEQ ID NO: 19. Another such example is LFₙ-Bcl-x_{L}, which includes the amino terminal portion (residues 1-255) of mature anthrax lethal factor (LF), coupled to residues 1-209 of Bcl-x_{L}. The nucleotide sequence of this fusion protein is shown in SEQ ID NO: 24, and the corresponding amino acid sequence in SEQ ID NO: 25.

In other examples, the therapeutic proteins comprise targeted cargo fusion proteins which have a targeting moiety that binds specifically to a target molecule on a target cell surface, and a cargo moiety that exerts a biological effect on the target cell. Targeting moeities may include antibodies, or cytokines or growth factors that target cytokine or growth factor receptors on cell surfaces. Cargo moieties may include toxins or pro-toxins, such as Pseudomonas exotoxin or Proaerolysin. In one example the targeted cargo protein is not the circularly permutted IL-4-Pseudomonas exotoxin PRX321 shown in FIG. 6.

The disclosure also provides methods of using therapeutically effective amounts of the disclosed compositions, for example to reduce an immune response in vivo to the therapeutic protein.

For example, the disclosed compositions can be administered to a subject in need of the modified PA, thereby reducing an immune response in the subject to the modified PA protein. In some examples, the subject is a human subject who has prostate cancer or benign prostatic hyperplasia (BPH) and the modified proaerolysin protein treats the prostate cancer or BPH. Thus, also provided are methods of treating prostate cancer (such as a localized or metastatic prostate cancer) and BPH using therapeutically effective amounts of the disclosed compositions.

In another example, the disclosed compositions can be administered to a subject in need of an apoptosis-modifying fusion protein thereby reducing an immune response in the subject to the apoptosis-modifying fusion protein. In some examples, the subject is a human subject who is need of a reduction of apoptotic damage that can be caused by neurodegenerative disorders (e.g., Alzheimer's disease, Huntington's disease, spinal-muscular atrophy), stroke episodes, and transient ischemic neuronal injury (e.g., spinal cord injury). The apoptosis-enhancing fusion proteins can be used to inhibit cell growth, for instance uncontrolled cellular proliferation.

Another aspect of the disclosure includes methods of decreasing immune responses to therapeutic proteins in subjects in need of treatment with the therapeutic proteins. Such methods can include administering one or more therapeutic proteins to a subject in a pharmaceutical composition that includes a sufficient amount of albumin to decrease the subject's immune responses to the therapeutic protein. In one example, the method includes decreasing the amount of antibody produced to the therapeutic protein by a subject following the administration of a therapeutic protein (such as a decrease of at least 10%, at least 20% at least 50%, at least 80%, or at least 90% relative to the absence of albumin). For example, the therapeutic protein compositions described herein can be combined with a sufficient amount of albumin prior to administration to a subject.

In a further aspect of the disclosure, the use of albumin containing formulations of therapeutic proteins (such as a modified proaerolysin (PA) protein, for example SEQ ID NO: 4 or SEQ ID NO: 28), results in compositions having increased viscosity which, when administered intraprostatically or intratumorally, results in a reduced amount of the therapeutic protein which flows out from the needle and prostate in conjunction with administration as compared to a composition which does not comprise human serum albumin. In certain embodiments, the reduction of the therapeautic protein which exits from the prostate is by at least 1%, at least 2%, at least 5%, at least 10%, at least 15%, at least 20%, at least 30%, at least 40%, at least 50%, at least 60%, at least 70%, at least 80%, or at least 90%.

In another aspect of the disclosure, the use of albumin-containing formulations of therapeutic proteins (such as a modified proaerolysin (PA) protein, for example SEQ ID NO: 4 or SEQ ID NO: 28), results in decreased adsorption of the therapeutic protein on vials such as storage, dilution, or mixing vial, or delivery devices such as syringes.

In certain embodiments, disclosed herein is a pharmaceutical composition, comprising:

albumin; and

a modified proaerolysin protein, wherein the modified proaerolysin protein comprises a prostate-specific protease cleavage site that replaces a proaerolysin furin cleavage site corresponding to amino acids 427-432 of SEQ ID NO: 2

In further embodiments, said albumin is human serum albumin.

In further embodiments, the albumin is a recombinant human serum albumin or a human serum albumin purified from human blood.

In further embodiments, said therapeutic protein is a modified proaerolysin protein selected from SEQ ID NO: 4 and SEQ ID NO: 28.

In further embodiments, said therapeutic protein is the protein of SEQ ID NO: 4.

In further embodiments, wherein said therapeutic protein is the protein of SEQ ID NO: 28.

In further embodiments, the molar ratio of the amount of human serum albumin to the amount of therapeutic protein is between 5:1 and 100,000:1.

In further embodiments, the molar ratio of the amount of human serum albumin to the amount of therapeutic protein is between 50:1 and 5,000:1.

In further embodiments, the amount of human serum albumin in the pharmaceutical composition is between 0.01 and 25% by weight.

In further embodiments, the amount of human serum albumin in the pharmaceutical composition is between 0.2 and 5% by weight.

In further embodiments, the amount of human serum albumin in the pharmaceutical composition is between 1.8 and 2.2% by weight.

In further embodiments, the amount of human serum albumin in the pharmaceutical composition is about 2% by weight.

In further embodiments, the amount of the protein of SEQ ID NO: 28 is between 2 and 4 μg/ml.

In further embodiments, the amount of the protein of SEQ ID NO: 28 is between 2.5 and 3.5 μg/ml.

In further embodiments, the amount of the protein of SEQ ID NO: 28 is between 2.8 and 3.2 μg/ml.

In further embodiments, the amount of the protein of SEQ ID NO: 28 is about 3 μg/ml.

In further embodiments, the amount of human serum albumin in the pharmaceutical composition is about 2% by weight.

In further embodiments, the composition comprises:
between 0.05 and 25.0 μg/ml of the protein of SEQ ID NO: 28;
between 1.5 and 2.5% human serum albumin by weight;
between 100 and 200 mM NaCl;
between 0.1 and 1.0 mM NaH₂PO₄;
between 5 and 15 mM Na₂HPO₄; and
between 0.5 and 1.5 mM disodium ethylenediaminetetraacetic acid.

In further embodiments, the composition comprises:
between 0.5 and 10.0 μg/ml of the protein of SEQ ID NO: 28;
between 1.8 and 2.2% human serum albumin by weight;
between 130 and 140 mM NaCl;
between 0.5 and 0.7 mM NaH₂PO₄;
between 7 and 9 mM Na₂HPO₄; and
between 0.8 and 1.0 mM disodium ethylenediaminetetraacetic acid.

In further embodiments, the composition comprises:
between 0.5 and 10.0 μg/ml of the protein of SEQ ID NO: 28;
about 2% human serum albumin by weight;
about 138 mM NaCl;
about 0.6 mM NaH₂PO₄;
about 8.5 mM Na₂HPO₄; and
about 0.92 mM disodium ethylenediaminetetraacetic acid.

In certain embodiments, disclosed herein is a kit, comprising:
a first container containing the therapeutic protein of SEQ ID NO: 28; and
a second container containing human serum albumin.

In further embodiments,
the first container contains a solution comprising about 300 μg/ml of the therapeutic protein of SEQ ID NO: 28;
the second container contains a solution comprising about 2% human serum albumin; and
further comprising instructions to transfer the solution from the first container into the second container.

In further embodiments, the second container contains a solution comprising:
between 0.5 and 10.0 μg/ml of the protein of SEQ ID NO: 28;
between 1.8 and 2.2% human serum albumin by weight;
between 130 and 140 mM NaCl;
between 0.5 and 0.7 mM NaH2PO4;
between 7 and 9 mM Na2HPO4; and
between 0.8 and 1.0 mM disodium ethylenediaminetetraacetic acid.

In further embodiments, the second container contains a solution comprising:
between 0.5 and 10.0 μg/ml of the protein of SEQ ID NO: 28;
about 2% human serum albumin by weight;
about 138 mM NaCl;
about 0.6 mM NaH₂PO₄;
about 8.5 mM Na₂HPO₄; and
about 0.92 mM disodium ethylenediaminetetraacetic acid.

In certain embodiments, disclosed herein is a method for treating benign prostatic hyperplasia in a subject comprising the administration of a therapeutically effective amount of a composition disclosed herein to a patient in need thereof.

In further embodiments, administration of the composition results in a reduction in prostate volume or weight or a reduction in the rate of prostate growth.

In further embodiments, the prostate size is reduced by at least 10%.

In further embodiments, the composition is administered intraprostatically.

In further embodiments, the amount of the protein of SEQ ID NO: 28 which exits from the prostate after administration is reduced in comparison with a composition which does not comprise human serum albumin.

In further embodiments, the amount of the protein of SEQ ID NO: 28 which exits from the prostate after administration is reduced by at least 1%, at least 2%, at least 5%, at least 10%, at least 15%, at least 20%, at least 30%, at least 40%, at least 50%, at least 60%, at least 70%, at least 80%, or at least 90%.

In further embodiments, the immune response is decreased in comparison with a composition which does not comprise human serum albumin.

In further embodiments, the immune response is an antibody response.

In further embodiments, the antibody response is decreased by at least 1%, at least 2%, at least 5%, at least 10%, at least 15%, at least 20%, at least 30%, at least 40%, at least 50%, at least 60%, at least 70%, at least 80%, or at least 90%.

In further embodiments, the antibody response is decreased by at least 90%.

In further embodiments, the antibody response is decreased 2-fold to 10-fold.

In certain embodiments, disclosed herein is a method for treating prostate cancer in a subject comprising the administration of a therapeutically effective amount of the composition disclosed herein to a patient in need thereof.

In further embodiments, the subject has a localized prostate tumor.

In further embodiments, the subject has metastatic prostate cancer.

In further embodiments, administration of the composition results in a reduction in prostate tumor volume.

In further embodiments, the prostate tumor volume is reduced by at least 10%.

In further embodiments, the composition is administered intraprostatically.

In further embodiments, the amount of the protein of SEQ ID NO: 28 which flows out from the needle and prostate in conjunction with administration is reduced in comparison with a composition which does not comprise human serum albumin.

In further embodiments, the amount of the protein of SEQ ID NO: 28 which flows out from the needle and prostate in conjunction with administration is reduced by at least 1%, at least 2%, at least 5%, at least 10%, at least 15%, at least 20%, at least 30%, at least 40%, at least 50%, at least 60%, at least 70%, at least 80%, or at least 90%.

In further embodiments, the immune response is decreased in comparison with a composition which does not comprise human serum albumin.

In further embodiments, the immune response is an antibody response.

In further embodiments, the antibody response is decreased by at least 1%, at least 2%, at least 5%, at least 10%, at least 15%, at least 20%, at least 30%, at least 40%, at least 50%, at least 60%, at least 70%, at least 80%, or at least 90%.

In further embodiments, the antibody response is decreased by at least 90%.

In further embodiments, the antibody response is decreased 2-fold to 10-fold.

In certain embodiments, disclosed herein is a method of decreasing an antibody response to a therapeutic protein of selected from SEQ ID NO: 4 and SEQ ID NO: 28 comprising administering the therapeutic protein to the subject in a pharmaceutical composition comprising a sufficient amount of albumin to decrease the subject's antibody response to the therapeutic protein.

In further embodiments, the antibody response is decreased by 10% to 90%.

In further embodiments, the antibody response is decreased by at least 90%.

In further embodiments, the antibody response is decreased 2-fold to 10-fold.

In further embodiments, the albumin is a human serum albumin.

In further embodiments, the albumin is a recombinant human serum albumin or a human serum albumin purified from human blood.

In further embodiments, the molar ratio of the amount of albumin to the amount of therapeutic protein is between 5:1 and 100,000:1 or between 50:1 and 5,000:1.

In further embodiments, the amount of albumin in the pharmaceutical composition is between 0.01 and 25% by weight, between 0.2 and 5% by weight, or 2% by weight.

In certain embodiments, disclosed herein is a method of decreasing an antibody response to a therapeutic protein in a subject in need of treatment therewith, comprising administering the therapeutic protein to the subject in a pharmaceutical composition comprising a sufficient amount of albumin to decrease the subject's antibody response to the therapeutic protein.

In further embodiments, the antibody response is decreased by 10% to 90%.

In further embodiments, the antibody response is decreased by at least 90%.

In further embodiments, the antibody response is decreased 2-fold to 10-fold.

In further embodiments, the albumin is a human serum albumin.

In further embodiments, the albumin is a recombinant human serum albumin or a human serum albumin purified from human blood.

In further embodiments, the molar ratio of the amount of albumin to the amount of therapeutic protein is between 5:1 and 100,000:1 or between 50:1 and 5,000:1.

In further embodiments, the amount of albumin in the pharmaceutical composition is between 0.01 and 25% by weight, between 0.2 and 5% by weight, or 2% by weight.

In further embodiments, the therapeutic protein is a targeted cargo protein, wherein the targeted cargo protein comprises:
a targeting moiety that specifically binds to a target displayed by a target cell, and
a cargo moiety that exerts a biological effect on the target cell.

In further embodiments, the targeted cargo protein comprises one or more cargo moieties selected from aerolysin, proaerolysin, bouganin, abrin, ricin, Pseudomonas exotoxin, cholera toxin, diphtheria toxin, tetanus toxin, neural thread protein and Bad.

In further embodiments, the targeted cargo protein comprises Pseudomonas exotoxin linked to circularly permuted IL-4, IL-2 linked to aerolysin, IL-2 linked to proaerolysin, IL4 linked to BAD, GMCSF linked to BAD, EGF linked to proaerolysin, anti-EpCAM antibody linked to Pseudomonas exotoxin, anti-EpCAM antibody linked to bouganin, anti-mesothelin antibody linked to Pseudomonas exotoxin, anti-CD22 antibody linked to Pseudomonas exotoxin, anti-CD22 antibody linked to RNase A, and anti-PSMA antibody linked to thapsigargin.

In further embodiments, the therapeutic protein comprises Pseudomonas exotoxin linked to circularly permuted IL-4 and the molar ratio of the amount of the therapeutic protein to the amount of albumin is 5:1 to 5000:1, such as 50:1.

In further embodiments, wherein the therapeutic protein comprises an apoptosis-modifying fusion protein comprising an inactive toxin protein domain, an apoptosis regulating protein domain, wherein the inactive toxin protein domain targets the fusion protein to the cell and is not biologically active.

In further embodiments, the apoptosis regulating protein domain comprises a Bcl-2 protein.

In further embodiments, the Bcl-2 protein is a pro-apoptotic protein selected from Bcl-xs, Bax, Bad, Bak, DIVA, Bak, Bik, Bim, Bid and Egl-1, or an anti-apoptotic protein selected from Bcl-xL, Mcl-1, Ced-9 and A1.

In further embodiments, the inactive toxin protein domain comprises a domain derived from diphtheria toxin, tetanus toxin or anthrax toxin.

In certain embodiments, disclosed herein is a pharmaceutical composition comprising:
albumin; and
a therapeutic protein selected from the group consisting of:
an apoptosis-modifying fusion protein comprising an inactive toxin protein domain and an apoptosis regulating protein domain, wherein the inactive toxin protein domain targets the fusion protein to the cell and is not biologically active, or a targeted cargo protein, comprising a targeting moiety that specifically binds to
a target displayed by a target cell, and a cargo moiety that exerts a biological effect on a target cell.

In further embodiments, the albumin is human serum albumin.

In further embodiments, the apoptosis regulating protein domain comprises a Bcl-2 protein domain.

In further embodiments, the Bcl-2 protein is Bcl-X_{L}.

In further embodiments, the Bcl-X_{L} comprises amino acid residues 1-209 of Bcl-X_{L}.

In further embodiments, the inactive toxin protein domain comprises an inactive anthrax toxin domain or an inactive diphtheria toxin domain.

In further embodiments, the inactive anthrax toxin domain comprises an amino terminal portion of mature anthrax lethal factor (LF).

In further embodiments, the amino terminal portion comprises amino acid residues 1-255 of mature anthrax LF.

In further embodiments, the therapeutic protein comprises at least 95% sequence identity to the amino acid sequence shown in SEQ ID NO: 26.

In further embodiments, the inactive diphtheria toxin domain comprises the translocation region (domain or sub-domain) of diphtheria toxin.

In further embodiments, the therapeutic protein comprises at least 95% sequence identity to the amino acid sequence shown in SEQ ID NO: 20 or 22.

In certain embodiments, disclosed herein is a method for modifying apoptosis in a target cell, comprising contacting the target cell with an amount of the composition disclosed herein, sufficient to inhibit apoptosis.

In further embodiments, apoptosis in the target cell is inhibited.

In further embodiments, the target cell is a neuron, a lymphocyte, a macrophage, an epithelial cell, or a stem cell.

In further embodiments, apoptosis in the target cell is enhanced.

In further embodiments, further comprising the step of co-administering an agent selected from the group consisting of a chemotherapeutic agent, an anti-inflammatory agent, an anti-viral agent, and an antibiotic agent.

In further embodiments, the target cell is a tumor cell, a cancer cell, a neoplasm cell, a hyper-proliferative cell, or an adipocyte.

In certain embodiments, disclosed herein is a method of reducing apoptosis in a subject after transient ischemic neuronal injury, comprising administering to the subject a therapeutically effective amount of a composition disclosed herein.

In further embodiments, the transient ischemic neuronal injury is a spinal cord injury.

The foregoing and other objects and features of the disclosure will become more apparent from the following detailed description, which proceeds with reference to the accompanying figures.

### BRIEF DESCRIPTION OF THE DRAWINGS

FIG. 1 is a graph showing antibody titers in rats receiving a single injection of PRX302 either intraprostatically (IP) or intravenously (IV) using an old formulation that did not include HSA.

FIG. 2 is a graph showing antibody titers in cynolmolgus monkeys receiving a single intraprostatic injection of PRX302 using an old formulation that did not include HSA.

FIG. 3 is a bar graph showing antibody titers in serum of patients 90 days post treatment with PRX302 in Phase I Prostate Cancer and BPH study and Phase IIa and IIb BPH studies. Old formulation is PRX302 without albumin, and new formulation is PRX302 with HSA.

FIG. 4 is a bar graph showing average antibody titers in Phase I and II studies with PRX302 (error bars indicate standard deviation). Range of total amount injected into the prostate for each group in parentheses.

FIG. 5 is a bar graph showing antibody titers in 3 patients from Phase IIa BPH study measured longitudinally over 360 days.

FIG. 6 is a schematic representation of the amino acid sequence of an exemplary tarted cargo protein, a circularly permuted IL-4-Pseudomonas exotoxin, known as PRX321. Disulfide bonds are indicated on the drawing.

### SEQUENCE LISTING

The nucleic and amino acid sequences listed in the accompanying sequence listing are shown using standard letter abbreviations for nucleotide bases, and three letter code for amino acids. Only one strand of each nucleic acid sequence is shown, but the complementary strand is understood as included by any reference to the displayed strand.

SEQ ID NOS: 1 and 2 show a wild-type proaerolysin cDNA and protein sequence, respectively.

SEQ ID NOS: 3 and 4 show the PRX302 cDNA and protein sequence, respectively, wherein the furin site of proaerolysin has been replaced with a PSA cleavage site. SEQ ID NO: 28 shows the protein sequence of SEQ ID NO: 4 with an N-terminal His tag.

SEQ ID NOS: 5-15 are exemplary PSA cleavage sites.

SEQ ID NO: 16 is a native luteinizing hormone releasing hormone (LHRH) protein sequence.

SEQ ID NO: 17 is a modified LHRH protein sequence.

SEQ ID NOs: 18 and 19 show the DNA coding sequence and corresponding amino acid sequence of Bcl-x_{L}-DTR.

SEQ ID NOs: 20 and 21 show the DNA coding sequence and corresponding amino acid sequence of Bad-DTTR.

SEQ ID NO: 22 shows the nucleotide sequence of the linker used to link Bcl-x_{L} to DTR in the fusion construct Bcl-x_{L}-DTR.

SEQ ID NO: 23 shows the amino acid sequence of the linker used to link Bcl-x_{L} to DTR to form Bcl-x_{L}-DTR.

SEQ ID NOs: 24 and 25 show the DNA coding sequence and corresponding amino acid sequence of LFₙ-Bcl-x_{L}.

SEQ ID NOs: 26 and 27 show the DNA coding sequence and corresponding amino acid sequence of PRX-321.

### DETAILED DESCRIPTION

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. For example, the term “a protein” includes single or plural proteins and can be considered equivalent to the phrase “at least one protein.” As used herein, the term “comprises” means “includes.” Thus, “comprising a protein” means “including a protein” without excluding other elements.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

It is further to be understood that all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references and GenBank accession numbers available on Oct. 22, 2010, are incorporated by reference.

To facilitate review of the various embodiments of the disclosure, the following explanations of abbreviations and terms are provided:
Ab: antibody
Ag: antigen
BPH: benign prostatic hyperplasia
DT: diphtheria toxin
DTR: diphtheria toxin receptor binding domain
DTT: diphtheria toxin translocation domain
DTTR: diphtheria toxin translocation and receptor binding domains
HSA: human serum albumin
LF: anthrax lethal factor
LFn: first 255 residues of anthrax lethal factor
PA: proaerolysin
PE: Pseudomonas exotoxin
PBS-EDTA: Phosphate-buffered saline-ethylenediamine tetraacetic acid
PSA: prostate specific antigen

Administer: To provide or give a subject an agent, such as a composition disclosed herein, by any effective route. Exemplary routes of administration include, but are not limited to, injection (such as intratumoral, intraprostatic, subcutaneous, intramuscular, intradermal, intraperitoneal, and intravenous), oral, sublingual, rectal, transdermal, intranasal, and inhalation routes. In particular examples, the compositions provided herein are administered intratumorally or intraprostatically, for example in a volume of 5 to 100 mL, such as 10 to 50 mL, for example 20 mL.

Aerolysin: A channel-forming toxin produced as an inactive protoxin called proaerolysin (PA) (wild-type PA is shown in SEQ ID NOS: 1 and 2). The PA protein contains many discrete functionalities that include a binding domain (approximately amino acids 1-83 of SEQ ID NO: 2), a toxin domain (approximately amino acids 84-426 of SEQ ID NO: 2), and a C-terminal inhibitory peptide domain (approximately amino acids 427-470 of SEQ ID NO: 2) that contains a protease activation site (amino acids 427-432 of SEQ ID NO: 2).

The binding domain recognizes and binds to glycophosphatidylinositol (GPI) membrane anchors, such as are found in Thy-1 on T lymphocytes, the PIGA gene product found in erythrocyte membranes and Prostate Stem Cell Antigen (PSCA). Most mammalian cells express GPI anchored proteins on their surfaces. The activation or proteolysis site within proaerolysin is a six amino acid sequence that is recognized as a proteolytic substrate by the furin family of proteases. PA is activated upon hydrolysis of a C-terminal inhibitory segment by furin. Activated aerolysin binds to GPI-anchored proteins in the cell membrane and forms a heptamer that inserts into the membrane producing well-defined channels of ˜17 Å. Channel formation leads to rapid cell death via necrosis. Wild-type aerolysin is toxic to mammalian cells, including erythrocytes, for example at 1 nanomolar or less.

Albumin Refers to albumin proteins, such as those found in the blood or serum. Serum albumin is a soluble, monomeric protein which comprises about one-half of the blood serum protein. Albumin functions primarily as a carrier protein for steroids, fatty acids, and thyroid hormones and plays a role in stabilizing extracellular fluid volume. Includes human serum albumin (HSA, OMIM 103600).

Albumin sequences are well-known in the art and are publically available, for example from GenBank. For example, GenBank Accession Nos. NP_00468 and AAA98797.1 provide exemplary human serum albumin protein sequences. In addition, Table 4 of U.S. Pat. No. 7,592,101 (Table 4 herein incorporated by reference) provides exemplary accession numbers for non-human serum albumin proteins available in GenBank. Albumin is also commercially available for example from Sigma-Aldrich (St. Louis, Mo.), Lee BioSolutions (St. Louis, Mo.) and Nova Biologics (Oceanside, Calif.). Recombinant human serum albumin is available from Novozymes Biopharma US Inc. (Cambridge, Mass.), GTC Biotheraeutics, Framingham Mass., Mitsubushi Welfarma, Osaka, Japan, and In Vitra, Junction City Kans. Human serum albumin that has been purified and is suitable for use in humans is available from Baxter Healthcare Corporation, Westlake Village, Calif. (Product Code #1500233).

Antibody (Ab): Immunoglobulin molecules and immunologically active portions of immunoglobulin molecules, i.e., molecules that contain an antigen binding site which specifically binds (immunoreacts with) an antigen. Exemplary antibodies are those that specifically bind to a therapeutic protein, such as a modified proaerolysin protein or an apoptosis-modifying fusion protein disclosed herein, such as antibodies (e.g., neutralizing antibodies) generated in response to administration of one or more therapeutically effective doses of a therapeutic protein (such as a modified proaerolysin protein or an apoptosis-modifying fusion protein) to a human.

A naturally occurring antibody (e.g., IgG) includes four polypeptide chains, two heavy (H) chains and two light (L) chains inter-connected by disulfide bonds. However, the antigen-binding function of an antibody can be performed by fragments of a naturally occurring antibody. Thus, these antigen-binding fragments are also intended to be designated by the term antibody. Examples of binding fragments encompassed within the term antibody include (i) an Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment (Ward et al., Nature 341:544-6, 1989) which consists of a VH domain; (v) an isolated complimentarity determining region (CDR); and (vi) an F(ab′)2 fragment, a bivalent fragment comprising two Fab fragments linked by a disulfide bridge at the hinge region.

Exemplary antibodies also include antibodies that specifically bind to a target protein (e.g., a cell surface receptor such as an IL-4 receptor). The term antibodies includes monoclonal antibodies, polyclonal antibodies as well as antibodies that have been humanized to render them less immunogenic. Methods of producing polyclonal and monoclonal antibodies are known to those of ordinary skill in the art, and many antibodies are available. See, e.g., Coligan, Current Protocols in Immunology Wiley/Greene, N Y, 1991; and Harlow and Lane, Antibodies: A Laboratory Manual Cold Spring Harbor Press, N Y, 1989; Stites et al., (eds.) Basic and Clinical Immunology (4th ed.) Lange Medical Publications, Los Altos, Calif., and references cited therein; Goding, Monoclonal Antibodies: Principles and Practice (2d ed.) Academic Press, New York, N.Y., 1986; and Kohler and Milstein, Nature 256: 495-497, 1975. Other suitable techniques for antibody preparation include selection of libraries of recombinant antibodies in phage or similar vectors. See, Huse et al., Science 246: 1275-1281, 1989; and Ward et al., Nature 341: 544-546, 1989.

Immunoglobulins and certain variants thereof are known and many have been prepared in recombinant cell culture (e.g., see U.S. Pat. No. 4,745,055; U.S. Pat. No. 4,444,487; WO 88/03565; EP 256,654; EP 120,694; EP 125,023; Faoulkner et al., Nature 298:286, 1982; Morrison, J. Immunol. 123:793, 1979; Morrison et al., Ann Rev. Immunol 2:239, 1984). Detailed methods for preparation of chimeric (humanized) antibodies can be found in U.S. Pat. No. 5,482,856. Additional details on humanization and other antibody production and engineering techniques can be found in Borrebaeck (ed), Antibody Engineering, 2nd Edition Freeman and Company, N Y, 1995; McCafferty et al., Antibody Engineering, A Practical Approach, IRL at Oxford Press, Oxford, England, 1996, and Paul Antibody Engineering Protocols Humana Press, Towata, N J, 1995.

In some examples, antibodies bind to their target with a binding constant that is at least 10³ M⁻¹ greater, 10⁴ M⁻¹ greater or 10⁵ M⁻¹ greater than a binding constant for other molecules in a sample. In some examples, a specific binding reagent (such as an antibody (e.g., monoclonal antibody) or fragments thereof) has an equilibrium constant (K_{d}) of 1 nM or less. For example, a specific binding agent may bind to a target protein with a binding affinity of at least about 0.1×10⁻⁸ M, at least about 0.3×10⁻⁸M, at least about 0.5×10⁻⁸M, at least about 0.75×10⁻⁸ M, at least about 1.0×10⁻⁸ M, at least about 1.3×10⁻⁸ M at least about 1.5×10⁻⁸M, or at least about 2.0×10⁻⁸ M. Kd values can, for example, be determined by competitive ELISA (enzyme-linked immunosorbant assay) or using a surface-plasmon resonance device such as the Biacore T100, which is available from Biacore, Inc., Piscataway, N.J.

“Specifically binds” refers to the ability of individual antibodies to specifically immunoreact with an antigen, such as PA or modified PA, relative to binding to unrelated proteins, such as non-PA proteins, for example albumin. For example, a Bcl-x_{L}-DTR-specific binding agent binds substantially only the Bcl-x_{L}-DTR protein in a specific preparation. As used herein, the term “Bcl-x_{L}-DTR-specific binding agent” includes Bcl-x_{L}-DTR antibodies and other agents that bind substantially only to a Bcl-x_{L}-DTR protein in that preparation.

The binding is a non-random binding reaction between an antibody molecule and an antigenic determinant of the T cell surface molecule. The desired binding specificity is typically determined from the reference point of the ability of the antibody to differentially bind the T cell surface molecule and an unrelated antigen, and therefore distinguish between two different antigens, particularly where the two antigens have unique epitopes. An antibody that specifically binds to a particular epitope is referred to as a “specific antibody”.

Antigen (Ag): A compound, composition, or substance that can stimulate the production of antibodies or a T cell response in an animal, including compositions (such as one that includes a therapeutic protein such as modified proaerolysin protein) that are injected or absorbed into an animal. An antigen reacts with the products of specific humoral or cellular immunity, including those induced by heterologous antigens, such as the disclosed antigens. “Epitope” or “antigenic determinant” refers to the region of an antigen to which B and/or T cells respond. In one embodiment, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and nuclear magnetic resonance.

Examples of antigens include, but are not limited to, peptides, lipids, polysaccharides, and nucleic acids containing antigenic determinants, such as those recognized by an immune cell. In some examples, an antigen includes a modified proaerolysin peptide or immunogenic fragment thereof.

Apoptosis-modifying ability: A protein has apoptosis-modifying ability if it is capable of modifying apoptosis in a cell. This ability is usually measurable, either in vivo or in vitro, using any routine apoptosis assays known in the art. Appropriate techniques include dye exclusion (e.g., Hoechst dye No. 33342), assaying for caspase activity, and TUNEL-staining. The specific ability of a protein (such as a fusion protein) to modify the apoptotic response of a cell to various apoptosis-inducing stimuli can be determined by running standard apoptosis assays in the absence of or presence of various concentrations of the proteins. The results of the assay are then compared, and can be reported for instance by presenting the percentage of apoptosis that occurs in the presence of the protein.

Apoptosis-modifying fusion protein: Proteins that have at least two domains fused together, at least one domain comprising a cell binding region capable of targeting the fusion protein to a target cell (the targeting or cell-binding domain), and at least one domain capable of modifying apoptosis in the target cell (the apoptosis-modifying domain). Apoptosis-modifying fusion proteins are further characterized by their ability to integrate into or otherwise cross a cellular membrane of the target cell when delivered extracellularly. An apoptosis-modifying fusion protein is considered functional if it targets to the correct target cell, and modifies an apoptotic response of that cell.

In general, the two domains of the disclosed fusions are genetically fused together, in that nucleic acid molecules that encode each protein domain are functionally linked together, for instance directly or through the use of a linker oligonucleotide, thereby producing a single fusion-encoding nucleic acid molecule. The translated product of such a fusion-encoding nucleic acid molecule is the apoptosis-modifying fusion protein.

Apoptosis-modifying fusion proteins can be named according to how they influence apoptosis in the target cell. For instance, an apoptosis-modifying fusion protein that inhibits apoptosis in the target cell can be referred to as an apoptosis-inhibiting fusion protein (e.g., Bcl-xL-DTR and LFn-Bcl-xL). Likewise, if the fusion protein enhances apoptosis in the target cell, it can be referred to as an apoptosis-enhancing fusion protein (e.g., Bad-DTTR). Specific apoptosis-modifying fusion proteins are usually named for the proteins from which domains are taken to form the fusion, or from the domains actually used. For instance, “Bcl-xL-DTR” (SEQ ID NOs: 18 and 19) consists of the entire Bcl-xL protein fused in frame to the receptor-binding domain of diphtheria toxin (DTR) via a short linker.

Bcl-2: A Bcl-2 protein is a protein from the Bcl-2 family of proteins and includes those proteins related to Bcl-2 by sequence homology, which affect apoptosis. The family includes Bcl-2, Bcl-x (both the long and short forms), Bax, and Bad. Additional members of the Bcl-2 family of proteins are known (Adams and Cory, Science 281:1322-1326, 1998). Sequences of such molecules are publicly available, for example on Genbank. For example, GenBank Accession Nos. CAA80661 and Z23115 provide Bcl-xL nucleic acid and protein sequences, respectively, and GenBank Accession Nos. CAG46733 and CR541935 provide Bad nucleic acid and protein sequences, respectively.

Molecules that are derived from proteins of the Bcl-2 family include fragments of such proteins (e.g., fragments of Bcl-xL or Bad), generated either by chemical (e.g., enzymatic) digestion or genetic engineering means. Such fragments may comprise nearly all of the native protein, with one or a few amino acids being genetically or chemically removed from the amino or carboxy terminal end of the protein, or genetically removed from an internal region of the sequence, such as a deletion of 1 to 10 amino acids.

Benign prostatic hyperplasia (BPH): The increase in the prostate size in middle-aged and elderly men. Also referred to as benign enlargement of the prostate. It is characterized by hyperplasia of prostatic stromal and epithelial cells, resulting in the formation of large, fairly discrete nodules in the periurethral region of the prostate.

Cancer or Tumor: A malignant neoplasm that has undergone characteristic anaplasia with loss of differentiation, increase rate of growth, invasion of surrounding tissue, and is capable of metastasis. A prostate cancer is a cancer that has its primary origin in prostate tissue. Residual cancer is cancer that remains in a subject after any form of treatment given to the subject to reduce or eradicate the cancer. Metastatic cancer is a cancer at one or more sites in the body other than the site of origin of the original (primary) cancer from which the metastatic cancer is derived.

Cargo Moiety: A peptide (e.g., protein fragment or full length protein) or other molecule that can function to significantly modulate a target cell. In some examples a cargo moiety can trigger cell death (e.g., apoptosis). In some examples, cargo moieties can inhibit apoptosis. Exemplary cargo moieties include toxins, such as toxins derived from plants, microorganisms, and animals. In one example, the cargo moiety is not a botulinum toxin. In other examples, cargo moieties are proteins that normally contribute to the control of cell life cycles, for example a cargo moieties can be any protein that triggers cell death, such as via apoptotic or non-apoptotic pathways. In some examples, the cargo moiety is not a protein, but another molecule that can function to significantly reduce or inhibit the growth of a cancer cell, such as thapsigargin. In some examples, a cargo moiety is activated by a tumor-associated protease, such as PSA. Exemplary cargo moieties, and exemplary GenBank accession numbers, are provided in Table 1, below. In addition to native cargo sequences, variant sequences can also be used, such as mutant sequences with greater biological activity than that of the native sequence.

TABLE 1
Exemplary cargo moiety sequences

| Cargo Moiety | Accession Numbers* and References |
|---|---|
| Aerolysin | ABR14715.1; ABR14714.1 |
| Proaerolysin | AAA21938.1; P09167.2; U.S. Pat. No. 7,282,476 |
| | (proaerolysin sequences therein herein incorporated |
| | by reference) |
| Bouganin | AAL35962 and SEQ ID NO: 9 in U.S. Pat. No. |
| | 6.737,511, as well as variant sequences provided in |
| | U.S. Pat. No. 7,339,031 and WO 2005/090579 |
| | (bouganin sequences therein herein incorporated by |
| | reference) |
| Pseudomonas | 1IKP A; AAB59097.1; AAF90003.1 (also see SEQ |
| exotoxin | ID NO: 1 of U.S. Pat. No. 6,011,002) |
| Bcl-2 pro-apoptotic | BAD: CAG46757; AAH01901.1; CAG46733.1; |
| proteins such as | and sequences provided in U.S. Pat. No. 6,737,511 |
| Bad and Bax, Bak, | BAX: CAE52909.1; AAO22992.1; EAW52418.1 |
| DIVA, Bcl-Xs, Bik, | |
| Bim, Bid and Egl-1 | |
| BcL-2 anti-apoptotic | Bcl-xL: CAA80661.1 |
| protiens such as | |
| Bcl-xL, Mcl-1, | |
| CED-9 and A1 | |
| Cholera toxin | BAA06291.1; ACF35010.1; BAA06288.1; as well |
| | as variant sequences provided in US patent |
| | application No. 61/058,872 (variant cholera toxin |
| | sequences therein herein incorporated by reference) |
| Abrin | CAA54139.1; 1908235A; 1406189A |
| Ricin | P02879.1 |
| Verotoxin | BAF36762.1; BAF36761.1 |
| Diphtheria toxin | U.S. Pat. No. 6,737,511 |
| Tetanus toxin | GI: 135624 |
| Botulinum toxin | A5HZZ9; YP_001253342.1 |
| Neural thread | U.S. Pat. No. 7,259,232 |
| protein | |
| Ribonuclease A | BAA05124.1; NP_937877.1; NP_115961.2; |
| | Q5GAN4.1; and sequences provided in PCT |
| | Publication No. WO 2007/041361 (rapLR1 |
| | sequences therein herein incorporated by reference) |

*GenBank Numbers are herein incorporated by reference, as well as their corresponding nucleic acid sequences.

Conservative substitution: One or more amino acid substitutions (for example 2, 5 or 10 residues) for amino acid residues having similar biochemical properties. Typically, conservative substitutions have little to no impact on the activity of a resulting polypeptide. For example, ideally, a therapeutic protein (such as a modified PA peptide or an apoptosis-modifying fusion protein) including one or more conservative substitutions retains the biological activity of the therapeutic protein (such as a modified PA peptide, targeted cargo protein, or an apoptosis-modifying fusion protein). A polypeptide can be produced to contain one or more conservative substitutions by manipulating the nucleotide sequence that encodes that polypeptide using, for example, standard procedures such as site-directed mutagenesis or PCR.

Substitutional variants are those in which at least one residue in the amino acid sequence has been removed and a different residue inserted in its place. Examples of amino acids which may be substituted for an original amino acid in a protein and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val. A therapeutic protein can include one or more such substitutions, such as 1, 2, 5, 8, or 10, such as 1 to 10 of such substitutions. In one example, such variants can be readily selected for additional testing by performing an assay (such as those described in Examples 2-5 of U.S. Pat. No. 7,282,476) to determine if the variant retains modified PA activity. In one example, such variants can be readily selected for additional testing by performing an assay (such as those described in U.S. Pat. No. 6,737,511) to determine if the variant retains apoptosis-modifying fusion protein activity.

Permissive substitutions are non-conservative amino acid substitutions, but also do not significantly alter proaerolysin activity. An example is substitution of Cys for Ala at position 300 of SEQ ID NO: 2 or 4 or 28.

Further information about conservative substitutions can be found in, among other locations in, Ben-Bassat et al., (J. Bacteriol. 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988), WO 00/67796 (Curd et al.) and in standard textbooks of genetics and molecular biology.

Control level: The level of a response in the absence or presence of a particular molecule, such as an immune response (e.g., generation of antibodies against a therapeutic protein such as a modified PA protein, targeted cargo protein, or an apoptosis-modifying fusion protein) in the absence or presence of albumin. In certain embodiments, a control level of immunogenicity can be measured in a cell or subject that has not been subjected, either directly or indirectly, to a treatment with a composition that includes albumin, but may have received a therapeutic dose of a therapeutic protein such as modified PA protein, targeted cargo protein, or apoptosis-modifying fusion protein. In some examples, a control level is a reference value or range of values expected under particular conditions, such as a reference value or range of values for the expected level of immunogenicity expected in the presence or absence of added albumin to a composition containing a therapeutic protein such as modified PA protein, targeted cargo protein, or apoptosis-modifying fusion protein.

Decrease: To reduce the quality, amount, or strength of something. In one example, a therapeutic composition that includes albumin decreases the immunogenicity of a therapeutic protein such as modified PA, targeted cargo protein, or apoptosis-modifying fusion protein in the composition when administered to a subject, for example as compared to the response in the absence of the albumin. In a particular example, a composition that includes a modified PA or apoptosis-modifying fusion protein and albumin decreases the immune response to the modified PA or apoptosis-modifying fusion protein in a human subject. In some examples such a decrease is evidenced by the production of therapeutic protein-specific antibodies, such as modified PA-specific antibodies or apoptosis-modifying fusion protein specific antibodies. In some examples, the decrease in immunogenicity of therapeutic protein-specific antibodies, such as modified PA-specific antibody, targeted cargo protein-specific antibody, or apoptosis-modifying fusion protein-specific antibody production, is at least 10%, at least 20%, at least 50%, or even at least 90%, relative to the immune response observed with a composition that includes the therapeutic protein (such as modified PA, targeted cargo protein, or apoptosis-modifying fusion protein) but no albumin. In other examples, decreases are expressed as a fold change, such as a decrease in immunogenicity of the therapeutic protein-specific antibody (such as modified PA-specific antibody, targeted cargo protein specific antibody, or apoptosis-modifying fusion protein specific antibody) production by at least 2-fold, at least 3-fold, at least 4-fold, at least 5-fold, at least 8-fold, at least 10-fold, or even at least 15 or 20-fold, relative to the immune response observed with a composition that includes the therapeutic protein (such as modified PA, targeted cargo protein, or apoptosis-modifying fusion protein) but no albumin. Such decreases can be measured using the methods disclosed herein.

Immune response: A response of a cell of the immune system, such as a B cell, T cell, or monocyte, to a stimulus. In one embodiment, the response is specific for a particular antigen (an “antigen-specific response”), such as a modified PA protein, apoptosis-modifying fusion protein, or immunogenic fragment thereof. In one embodiment, an immune response is a T cell response, such as a CD4+ response or a CD8+ response. In another embodiment, the response is a B cell response, and results in the production of specific antibodies (such as those that specifically bind to a modified PA protein or an apoptosis-modifying fusion protein).

Immunogenic protein: A protein or a portion thereof that is capable of inducing an immune response in a mammal, such as a mammal with prostate cancer or BPH who is administered the protein (such as a modified PA). For example, such peptides can include a sequence such that the peptide will bind an MHC molecule and induce a cytotoxic T lymphocyte (“CTL”) response, or a B cell response (e.g., antibody production) against the antigen from which the immunogenic peptide is derived.

In some examples, an immunogenic polypeptide is a modified PA protein provided herein, such as SEQ ID NO: 4, capable of inducing an immune response in a mammal, such as a mammal with prostate cancer or BPH. In some examples, repeated administration of a modified PA protein results in an undesired immune response to the modified PA protein.

In some examples an immunogenic polypeptide is an apoptosis-modifying fusion protein, such as SEQ ID NO: 19, capable of inducing an immune response in a mammal, such as a mammal having a disorder that would benefit from increasing or decreasing apoptosis.

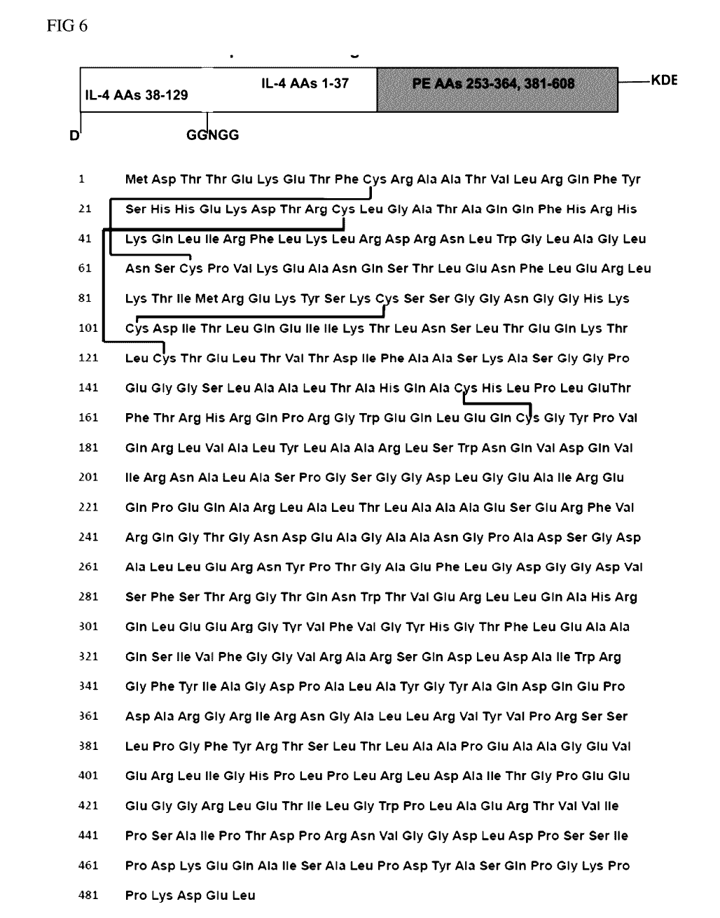

In some examples, an immunogenic polypeptide is a targeted cargo protein, such as a circularly permuted IL-4-pseudomonas exotoxin (PRX321) shown in FIG. 6.

In some examples, the immunogenic polypeptide contains non-human derived sequence of amino acids, such as toxin sequences.

Immunogenicity: The ability of an antigen, such as a therapeutic protein (e.g., a modified PA protein or apoptosis-modifying fusion protein), to induce a humoral or cell-mediated immune response.

Isolated: An “isolated” biological component (such as a nucleic acid molecule or protein) has been substantially separated or purified away from other biological components in the cell of the organism in which the component naturally occurs (e.g., other chromosomal and extrachromosomal DNA and RNA). Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

An isolated cell is one which has been substantially separated or purified away from other biological components of the organism in which the cell naturally occurs.

Linker: A peptide, usually between two and 150 amino acid residues in length, which serves to join two protein domains in a multi-domain fusion protein, such as an apoptosis-modifying fusion protein. Peptide linkers are generally encoded for by a corresponding oligonucleotide linker. This can be genetically fused, in frame, between the nucleotides that encode the domains of a fusion protein.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically Acceptable Carriers: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic agents, such as one or more compositions that include albumin and modified PA, targeted cargo protein, or an apoptosis-modifying fusion protein.

In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate, sodium lactate, potassium chloride, calcium chloride, and triethanolamine oleate. In one example, the pharmaceutically acceptable carrier is PBS/EDTA buffer at about pH 7.4, such as 10 mM PBS/EDTA at pH 7.4.

Proaerolysin: The inactive protoxin of aerolysin. The cDNA and protein of a wild-type or native proaerolysin are shown in SEQ ID NOS: 1 and 2, respectively.

In one example, a variant or modified proaerolysin molecule includes a prostate-specific protease cleavage site, such as a PSA-specific cleavage site, which permits activation of the variant PA in the presence of a prostate-specific protease such as PSA, PMSA, or HK2. In one example, a prostate-specific protease cleavage site is inserted into the native furin cleavage site of PA (e.g., amino acids 427-432 of SEQ ID NO: 2), such that PA is activated in the presence of a prostate-specific protease, but not furin. Alternatively, the furin cleavage site can be functionally deleted using mutagenesis of the six amino acid sequence, and insertion of a prostate-specific protease cleavage sequence. In another example, a variant PA molecule further includes deletion or substitution of one or more, such as at least two, of the native PA amino acids. In yet another example a variant PA molecule further includes another molecule (such as an antibody or peptide) linked or added to (or within) the variant PA molecule. In another example, a variant PA molecule includes a prostate-tissue specific binding domain.

In another example, a modified PA molecule further includes a functionally deleted binding domain (e.g., about amino acids 1-83 of SEQ ID NO: 2). Functional deletions can be made using any method known in the art, such as deletions, insertions, mutations, or substitutions. Examples include, but are not limited to deleting the entire binding domain (or portions thereof), or introduction of point mutations (such as those described above), which result in a binding domain with decreased function. For example, a PA molecule which has a functionally deleted binding domain (and no binding sequence substituted therefor), will have a decreased ability to accumulate in a cell membrane, and therefore lyse cells at a slower rate than a wild-type PA sequence. Also disclosed are modified PA molecules in which the native binding domain is functionally deleted and replaced with a prostate-tissue specific binding domain as described below.

Modified PA activity is the ability of a modified PA protein to lyse cells (such as prostate cells), for example thereby reducing signs or symptoms of BPH or prostate cancer. Cells include, but are not limited to prostate-specific protease secreting cells, such as PSA-secreting cells, such as prostate cancer cells, such as slow-proliferating prostate cancer cells. In one example, modified PA activity is said to be enhanced when modified PA proteins, when contacted with a PSA-secreting cell (such as a prostate cancer cell), promote lysis and death of the cell, for example by at least 10%, or for example by at least 25%, 50%, 100%, 200% or even 500%, when compared to lysis of a non-PSA producing cell. In other examples, modified PA activity is said to be enhanced when modified PA proteins, when contacted with a prostate tumor cell or prostate cell, decrease prostate cell or tumor volume, for example by at least 10% for example by at least 20%, 30%, 40%, 50%, 60%, 70%, 75%, 80%, 85%, 90%, 95%, 98%, or even 100% (complete elimination of the cell or tumor). In other examples, modified PA activity is said to be enhanced when modified PA proteins, when contacted with a normal prostate cell, promote lysis and death of the normal prostate cell, for example by at least 10%, or for example by at least 25%, 50%, 100%, 200% or even 500%, when compared to lysis of a non-prostate cell (such as a normal lung, spleen, or blood cell). In other examples, modified PA activity is said to be enhanced when modified PA proteins, when contacted with a prostate, decrease prostate volume or weight, for example by at least 10% for example by at least 20%, 30%, 40%, 50%, 60%, 70%, 75%, 80%, 85%, or 90%.

Assays which can be used to determine if a protein has modified PA activity are described in U.S. Pat. No. 7,282,476 (herein incorporated by reference, e.g., see Examples 2-5 and 9) and U.S. patent application Ser. No. 11/921,964. For example, a modified PA peptide can be assessed for its ability to specifically lyse PSA-producing cells, lyse normal prostate cells, decrease prostate volume or weight, attenuate further growth of the prostate, be stable in human plasma, be an efficient substrate for the enzymatic activity of PSA. Functional protein activity could be detected by the preferential lysis of PSA-producing cells versus non-PSA-producing cells, decreasing prostate tumor volume, having a decreased toxicity when compared to wild-type PA, and having an increased stability in blood when compared to wild-type PA.

Similar assays can be used to determine if any modified PA agent disclosed herein can decrease tumor volume (such as a prostate tumor) and specifically lyse PSA-producing cells, or use normal prostate cells (but not other normal cells such as spleen or lung cells). For example, the modified PA peptide shown in SEQ ID NO: 4 and 28 decreases prostate tumor volume and weight and can decrease or attenuate further growth of the prostate gland.

Prostate-specific protease cleavage site: A sequence of amino acids which is recognized and specifically and efficiently hydrolyzed (cleaved) by a prostate-specific protease. Examples include, but are not limited to a PSA-specific cleavage site, a PSMA-specific cleavage site and an HK2-specific cleavage site.

PRX302: A modified proaerolysin where the furin site of proaerolysin has been replaced with a PSA-specific cleavage site. SEQ ID NOS: 3 and 4 show the PRX302 cDNA and protein sequence, respectively. SEQ ID NO: 28 shows the protein sequence of SEQ ID NO: 4 with an N-terminal His tag. The term “PRX302” includes the proteins of both SEQ ID NO: 4 and SEQ ID NO: 28.

A PSA-specific cleavage site is a sequence of amino acids which is recognized and specifically and efficiently hydrolyzed (cleaved) by prostate specific antigen (PSA). Such peptide sequences can be introduced into other molecules, such as PA, to produce prodrugs that are activated by PSA. Upon activation of the modified PA by PSA, PA is activated and can exert its cytotoxicity. Examples of PSA-specific cleavage sites, include, but are not limited to are those shown in SEQ ID NOS: 5-15, those disclosed in U.S. Pat. No. 5,866,679 to DeFeo-Jones et al., U.S. Pat. No. 5,948,750 to Garsky et al., U.S. Pat. No. 5,998,362 to Feng et al., U.S. Pat. No. 6,265,540 to Isaacs et al., U.S. Pat. No. 6,368,598 to D'Amico et al., and U.S. Pat. No. 6,391,305 to Feng et al. (all herein incorporated by reference).

Particular examples of PSMA-specific cleavage sites can be found in WO/0243773 to Isaacs and Denmeade (herein incorporated by reference). Particular examples of HK2-specific cleavage sites are disclosed in WO/0109165 to Denmeade et al. (herein incorporated by reference).

Prostate tissue-specific binding domain: A molecule, such as a peptide ligand, toxin, or antibody, which has a higher specificity for prostate cells than for other cell types. In one example, a prostate tissue specific binding domain has a lower K_{D} in prostate tissue or cells than in other cell types, (i.e., binds selectively to prostate tissues as compared to other normal tissues of the subject), for example at least a 10-fold lower K_{D}, such as an at least 20-, 50-, 75-, 100- or even 200-fold lower K_{ID}. Such sequences can be used to target an agent, such as a modified PA molecule, to the prostate. Examples include, but are not limited to: antibodies which recognize proteins that are relatively prostate-specific such as PSA, PSMA, hK2, prostasin, and hepsin; ligands which have prostate-selective receptors such as natural and synthetic luteinizing hormone releasing hormone (LHRH); and endothelin (binding to cognate endothelin receptor).

Purified: The term purified does not require absolute purity; rather, it is intended as a relative term. Thus, for example, a purified protein preparation is one in which the protein is more enriched than the protein is in its generative environment, for instance within a cell or in a biochemical reaction chamber. In one example, a preparation of protein is purified such that the protein represents at least 50% of the total protein content of the preparation. More purified preparations will have a protein of interest that represents at least 60%, 70%, 80% or 90% of the total protein content.

Sample: Biological samples containing genomic DNA, cDNA, RNA, or protein obtained from the cells of a subject, such as those present in peripheral blood, urine, saliva, semen, tissue biopsy, surgical specimen, fine needle aspriates, amniocentesis samples and autopsy material. In one example, a sample includes prostate cells obtained from a subject. In another example, a sample is a serum sample.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For example, a modified PA protein having at least 75%, at least 80%, at least 85%, at least 90%, at least 95%, at least 98%, or at least 99% sequence identity to SEQ ID NO: 4 or 28 can be used in the compositions and methods provided herein. In another example, an apoptosis-modifying fusion protein having at least 75%, at least 80%, at least 85%, at least 90%, at least 95%, at least 98%, or at least 99% sequence identity to SEQ ID NO: 19, 21 or 25 can be used in the compositions and methods provided herein.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, 99% sequence identity.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 60%, 70%, 80%, 90%, 95%, 98%, or 99% sequence identity determined by this method. For example, a modified PA nucleic acid sequence having at least 75%, at least 80%, at least 85%, at least 90%, at least 95%, at least 98%, or at least 99% sequence identity to SEQ ID NO: 3 can be used to generate the modified PA proteins disclosed herein. In another example, a nucleic acid molecule encoding an apoptosis-modifying fusion protein having at least 75%, at least 80%, at least 85%, at least 90%, at least 95%, at least 98%, or at least 99% sequence identity to SEQ ID NO: 18, 20 or 24 can be used to express an apoptosis-modifying fusion protein.

Sufficient amount: An amount that permits or provides the desired activity. In one example, a sufficient amount of albumin is added to a formulation of a therapeutic protein (such as PRX-302 or PRX-321) to reduce the immune response to the therapeutic proteins to a desired level. In one example, a sufficient amount of albumin is in the range of 50 to 5000 times the amount of the therapeutic protein on a molar basis, so that for every mole of therapeutic protein there are 50 to 5000 moles of albumin.

Subject: Living multicellular vertebrate organisms, a category which includes human and other mammalian veterinary subjects that require decrease in the immune response to a therapeutic peptide, such as a modified PA peptide (such as a subject with prostate cancer or BPH), targeted cargo protein, or an apoptosis-modifying fusion protein. Exemplary subjects include primates, such as humans and monkeys, as well as rodents, such as rats, rabbits, and mice.

Targeted Cargo Protein: Any protein that binds specifically to a target cell and exerts a biological effect on the target cell. In some examples, targeted cargo proteins include a targeting moiety and a cargo moiety, the targeting moiety specifically binds with a cancer cell and the cargo moiety significantly exerts an effect by reducing or inhibiting the growth of the cancer cell or killing the cancer cells. Because in some examples the cargo moiety is not a protein, such as a chemotherapeutic agent, and in some examples the targeting moiety is not a protein, the targeted cargo protein in some examples is not actually a protein. Numerous targeted cargo proteins and methods of making them are provided in WO2010/031185 which is hereby incorporated by reference in its entirety. Some specific examples of useful targeted cargo proteins are indicated by an “X” in Table 2.

TABLE 2
Exemplary targeted cargo proteins

| | Targeting Moiety | | | | | | | | |
|---|---|---|---|---|---|---|---|---|---|
| Cargo Moiety | Mesothelin | PSMA | CD22 | EpCAM | IL-2 | IL-4 | EGF | GMCSF | Tenascin |
| Aerolysin | | | | | X | | | | |
| Proaerolysin | | | | | X | | X | | |
| Pseudomonas | X | | X | X | | X | X | | X |
| exotoxin | | | | | | | | | |
| BAD | | | | | | X | | X | |
| Bouganin | | | | X | | | | | X |
| RNAseA | | | X | | | | | | |
| Thapsigarin | | X | | | | | | | |

Particularly useful targeted cargo proteins include IL-2-aerolysin (see WO 2007/140618), IL-2-proaerolysin (see WO 2007/140618), EGF-proaerolysin, IL-4-BAD, anti-EpCAM-PE, anti-EpCAM-bouganin, GMCSF-BAD, anti-mesothelin antibody-PE, anti-CD22-PE, anti-CD22-RNase A, circularly permuted IL4-PE (FIG. 6, U.S. Pat. No. 6,011,002) and anti-PSMA-thapsigargin, antibody to acetylcholine receptor-ricin (U.S. Pat. No. 6,780,413).

Targeting moiety: Any compound that specifically binds to a molecule (herein referred to as a target) displayed by a cell, for example a targeting moiety can be an antibody that binds to a target (e.g., receptor), a ligand (e.g., a cytokine or growth factor) that binds to a receptor, a permuted ligand that binds to a receptor, or a peptide sequence sensitive to cleavage by a tumor-associated protease. In some examples, a targeting moiety is activated by a tumor-associated protease, such as PSA. Typically, targeting moieties selectively bind to one type of cell displaying a target more effectively than they bind to other types of cells that do not display the target. Targeting moieties can be chosen to selectively bind to subsets of tumor cells, such as cancer cells or immune cells, such as lymphocytes. Targeting moieties include specific binding agents such as antibodies, natural ligands of the target on the cell, such as IL-4, derivatives of such natural ligands, and immunoglobulin A. In some examples, the targeting moiety is not biologically active (e.g., cannot activate a receptor), but retains the ability to bind to the target and thus direct the targeted cargo protein to the appropriate cells. Other exemplary targeting moieties include the protein (not yet fully characterized) which binds to the 8H9 monoclonal antibody (see WO 2004/050849).

Table 3 provides information relating to the sequences of exemplary natural ligands as well as other antigens that can be used as targeting moieties. In some examples, circular permuted ligands, such as circular permuted IL-4, can be used to bind target cells. As additional research is performed, new targets will be identified. These additional markers can be used as targets for binding to targeting moieties and targeted cargo proteins can be made to inhibit the growth of (or kill) cells displaying such ligands. One of ordinary skill in the art will appreciate that once a marker is known, standard methods of making antibodies to the identified marker can be used to make targeting moieties specific for the marker, thus, allowing for the development of a specific targeted cargo protein.

TABLE 3
Exemplary targeting moiety sequences

| Receptor or Antigen | |
|---|---|
| to be Targeted | Accession Number* or reference |
| EGF | NP_001954; EAX06257.1; AAR84237.1 |
| EpCAM | NP_002345; NP_032558.2; NP_612550.1 |
| IL-2 | CAA07317; AAB46883.1; NP_000577.2 |
| IL-3 | AAC08706.1; AAA99502.1; CAE45598.1 |
| IL-4 | AAH70123; CAA57444.1; AAH67515.1 (also see |
| | SEQ ID NO: 2 and various circularly permuted |
| | ligands in U.S. Pat. No. 6,011,002) |
| IL-5 | NP_000870.1; CAA01794.1; P32927.2 |
| IL-13 | AAH96141.2; AAH96138.1; AAH96139.1 |
| GMCSF | P04141.1; AAI13925.1; AAI08725.1 |
| Tenascin | AAA36728.1; CAA39628.1; NP_002151.2 |
| Mesothelin | CAC37289.1; ABW03459.1; AAH09272.1; |
| | AAH03512.1; as well as the mesothelins disclosed |
| | in U.S Pat. Nos. 7,081,518 and 6,051,405 |
| | (mesothelin sequences therein herein incorporated |
| | by reference) |
| CD22 | BAA36575.1; BAA36576.1; BAA36567.1 |
| PSMA (also known | ABO93402.2; AAC83972.1; NP_001014986.1; |
| as folate hydrolase) | NP_004467.1 |
| nicotinic | U.S. Pat. No. 6,780,413 |
| acetylcholine | |
| receptor (nAchR) | |

*GenBank Numbers are herein incorporated by reference, as well as their corresponding nucleic acid sequences.

Antibodies directed to such targets can be used as targeting moieties as well as the natural ligands of the targets and derivatives thereof.

Therapeutically Effective Amount: A quantity sufficient to achieve a desired biological effect in a subject being treated.

In one example it is an amount that is effective to decrease the size (e.g., volume), growth, side effects and/or metastasis of prostate cancer or an amount that is effect to decrease the size (e.g., volume or weight) of a prostate and/or other undesirable effects of BPH. When administered to a subject, an amount will generally be used that will achieve target tissue concentrations shown to achieve a desired in vitro or in vivo effect.

In one example, it is an amount sufficient in vivo to decrease the symptoms or effects of prostate cancer, such as the size or growth of the tumor, or the symptoms or effects of BPH (such as prostate size, volume or weight). In particular examples, it is an amount effective to decrease the size of a prostate tumor and/or prostate metastasis by at least 30%, at least 40%, at least 50%, at least 70%, at least 80%, at least 90%, at least 95%, at least 99% or even 100% (complete elimination of the tumor). In another example, it is an amount that can decrease the size, volume, or weight of a prostate by at least 10%, at least 20%, at least 30%, at least 40%, at least 50%, at least 75%, or even at least 90%.

In one example, it is an amount of apoptosis-modifying fusion protein sufficient to measurably inhibit or enhance apoptosis in a target cell in vivo. In particular examples, it is an amount effective to increase or decrease apoptosis by at least 10%, at least 30%, at least 40%, at least 50%, at least 70%, at least 80%, at least 90%, at least 95%, at least 99% or even 100%.

An effective amount of the compositions provided herein can be administered in a single dose, or in several doses, for example daily, during a course of treatment. However, the effective amount of will be dependent on the subject being treated, the severity and type of the condition being treated, and the manner of administration. For example, a therapeutically effective amount of modified PA can vary from about 0.1 to 1000 μg/kg of body weight, 1 to 500 μg/kg of body weight, 10 to 200 μg/kg of body weight, or 30 to 60 μg/kg of body weight if administered intraprostatically for the treatment of BPH, or 5 to 10 times these amounts if administered intratumorally for the treatment of prostate cancer. In one example, a therapeutically effective amount of apoptosis-modifying fusion protein can vary from about 0.01 mg/kg body weight to about 1 g/kg body weight.

In another example, a circularly permutted IL-4-Pseudomonas exotoxin as shown in FIG. 6 may be administered locally at a dosage of 90 μg into a tumor mass, administered by infusiton in a total volume of 60 ml.

Therapeutic protein(s): Macromolecules that include proteins, polypeptides, antibodies, peptides or fragments or varients thereof having one or more therapeutic and/or biological activities. Therapeutic proteins can include non-peptide components, such as DNA or RNA or small molecules. In some examples, the therapeutic proteins are at least partly non-human. In some embodiments, the therapeutic proteins comprise a toxin moiety. However, therapeutic proteins used in the methods and compositions provided herein may have been modified to make them less immunogenic to humans (for example, humanized antibodies, or modified Pseudomonas exotoxins (PE) having reduced immunogenicity compared to native PE as described in US Patent publication 2009/0142341). Some therapeutic proteins have been modified to reduce immunogenicity by removing antigenic epitopes or by coupling to molecules such as polyethylene glycol (PEG). However, the therapeutic proteins for use in the compositions and methods provided herein are immunogenic proteins. Exemplary therapeutic proteins include targeted cargo proteins modified-PA proteins, and apoptosis-modifying fusion proteins.

Treatment: Refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease.

For example, treatment of a subject with prostate cancer can include one or more of reducing the volume or size of a tumor, reducing metastasis of the tumor, slowing growth of the tumor, reducing PSA blood values, and the like. Such a reduction may be relative to no treatment (such as no administration of a modified PA protein).

For example, treatment of a subject with BPH can include reducing one or more of symptoms of BPH, such as one or more of: size of the prostate, volume of the prostate, weight of the prostate, frequent urge to urinate, passing only small amounts of urine, a burning sensation when urinating (dysuria), difficulty starting urination, interrupted flow (urinating in waves rather than a steady stream), weaker-than-normal urine flow, dribbling after urinating, excessive urinating at night (nocturia), sensation of not completely emptying the bladder, pain or discomfort in the lower back, in the area between the testicles and anus, in the lower abdomen or upper thighs, or above the pubic area, pain or vague discomfort during or after ejaculation, and pain in the tip of the penis. Such a reduction in these symptoms may be relative to no treatment (such as no administration of a modified PA protein). In some examples, further growth of the prostate is attenuated.

### Compositions for Reducing Immunogenicity

The disclosure provides pharmaceutical compositions for decreasing an immune response to a therapeutic protein in a subject in need of treatment therewith. Such compositions can include a therapeutically effective amount of the therapeutic protein; and a sufficient amount of albumin to decrease the subject's immune response to the therapeutic protein. Exemplary therapeutic proteins include modified PA proteins, apoptosis modifying proteins, and targeted cargo proteins. For example, the disclosure provides compositions that include a sufficient amount of albumin and a therapeutic protein, such as a modified proaerolysin protein, wherein the modified proaerolysin protein comprises a prostate-specific protease cleavage site that replaces a proaerolysin furin cleavage site corresponding to amino acids 427-432 of SEQ ID NO: 2, an apoptosis-modifying fusion protein comprising an inactive toxin protein domain and an apoptosis regulating protein domain, wherein the inactive toxin protein domain targets the fusion protein to the cell and is not biologically active, or a targeted cargo protein, comprising a targeting moiety that specifically binds to a target displayed by a target cell, and a cargo moiety that exerts a biological effect on a target cell, wherein the albumin reduces the immunogenicity of the therapeutic protein.

Bacterial toxins, such as aerolysin produced by Aeromonas hydrophilia and α-hemolysin produced by Staph aureus, are beta-sheet proteins that oligomerize in the plasma membrane to produce pores that lead to rapid cytolytic cell death. Pore formation physically disrupts the cell membranes, and results in death of cells in all phases of the cell cycle, including non-proliferating cells. Although wild-type aerolysin kills cells indiscriminately, an inactive protoxin form of aerolysin (a modified PA) can be targeted to, and activated by, prostate specific proteins. One advantage of the modified PA molecules for treatment of localized and metastatic prostate cancer or BPH is that it combines a proliferation independent therapy with prostate-specific drug delivery, resulting in minimal side effects to patients. However, it has been observed that administration of a modified PA protein results in undesired immune responses (e.g., neutralizing antibody production) in the patient which can reduce the efficacy of repeated administration. Thus, provided herein are compositions that can reduce or eliminate such undesired effects.

Compositions are provided herein that include albumin and a therapeutic protein, such as a modified proaerolysin (PA) protein, wherein the albumin reduces the immunogenicity of the therapeutic protein, such as modified PA protein. The albumin and therapeutic protein, such as modified PA protein are not a fusion protein (e.g., albumin is not fused to the N- or C-terminus of the modified PA), but instead are present in the same composition as individual proteins. However, without wishing to be bound to a particular theory, it is possible that at least some albumin “sticks” or otherwise binds to the therapeutic protein, such as modified PA protein. In some examples, the modified PA protein includes a prostate-specific protease cleavage site that functionally replaces a PA furin cleavage site corresponding to amino acids 427-432 of SEQ ID NO: 2.

Also provided are compositions that include albumin and an apoptosis-modifying fusion protein comprising an inactive toxin protein domain and an apoptosis regulating protein domain, wherein the inactive toxin protein domain targets the fusion protein to the cell and is not biologically active, wherein the albumin reduces the immunogenicity of the fusion protein. The albumin and apoptosis-modifying fusion protein are not a fusion protein (e.g., albumin is not fused to the N- or C-terminus of the apoptosis-modifying fusion protein), but instead are present in the same composition as individual proteins. However, without wishing to be bound to a particular theory, it is possible that at least some albumin “sticks” or otherwise binds to the apoptosis-modifying fusion protein. In some examples, the apoptosis-modifying fusion protein has a sequence that comprises at least 90%, at least 95%, at least 98%, or at least 99% sequence identity to SEQ ID NO: 19, 21 or 25.

Albumin proteins that can be used in the disclosed compositions are conventional. Such proteins are well known in the art and are commercially available. They may be either purified from serum or made recombinantly. In one example, the albumin is a human serum albumin.

In some examples, the composition includes a greater amount of albumin than the therapeutic protein (such as modified PA protein or apoptosis-modifying fusion protein) by the molar ratio of albumin to therapeutic protein. In some examples, the amount of albumin exceeds the therapeutic protein in the composition by at least 10-fold, such as at least 50-fold, at least 100-fold, at least 500-fold, at least 1000-fold, at least 2500-fold, at least 5000-fold, at least 6000-fold, at least 10,000-fold, at least 20,000-fold, at least 50,000-fold, at least 100,00-fold by molar ratio, such as 50- to 50,000 fold, 50- to 25,000-fold, or 100- to 10,000-fold by molar ratio. In some embodiments, the molar ratio of albumin to therapeutic protein is in the range of 50:1 to 5000:1. In one example, the molar ratio of the modified PA therapeutic protein PRX302 to albumin is in the range of 500:1 to 50,000:1 or about 5000:1 when used in the treatment of BPH. In another example, the molar ratio of modified PA therapeutic protein PRX 302 to albumin is in the range of 50:1 to abut 5000:1, or about 500:1 when used for the treatment of prostate cancer. In another example, the molar ratio of albumin to circularly permuted IL-4-Pseudomonas exotoxin (PRX 321; see U.S. Pat. No. 6,011,002) is in the range of 500:1 to 50,000:1, or about 5000:1.

The disclosed compositions can be formulated in conventional pharmaceutically acceptable carriers (vehicles) such as those found in Remington's Pharmaceutical Sciences, by E. W. Martin, Mack Publishing Co., Easton, Pa., 15th Edition (1975). The term “pharmaceutically acceptable carrier” refers to a carrier medium which does not interfere with the effectiveness of the biological activity of the active ingredients and which is not toxic to the host or patient. Representative examples are provided below. Pharmaceutical compositions can include albumin and one or more therapeutic proteins (such as one or more modified PA proteins, one or more targeted cargo proteins, or one or more apoptosis-modifying fusion proteins) and one or more non-toxic pharmaceutically acceptable carriers, diluents, or excipients. If desired, other active ingredients may be included in the compositions. As discussed herein, compositions that include modified PA proteins are suitable for use in the treatment of prostate cancer or BPH, and can reduce the immunogenicity of modified PA protein in the composition. Furthermore, compositions that include apoptosis-modifying fusion proteins are suitable for use in the treatment of disorders where increased or decrease apoptosis is desired, and can reduce the immunogenicity of apoptosis-modifying fusion proteins in the composition. In one example, the composition is liquid solution.

The pharmaceutical compositions may include, for example, from about 0.02% to about 25% by weight albumin (such as HSA), for example 0.5% to 10%, 0.5% to 5%, 1% to 3%, such as 2% by weight albumin. Concentration of the therapeutic protein, such as a modified PA protein, in the final formulation can be at least 0.1 μg/mL, such as at least 1 μg/mL, or at least 10 μg/mL, such as 0.1 to 30 μg/mL, 0.1 to 10 μg/mL, 1 to 5 μg/ml, such as 3 μg/mL. Concentration of a targeted cargo protein or an apoptosis-modifying fusion protein in the final formulation can be at least 0.1 μg/mL, such as at least 1 μg/mL, or at least 10 μg/mL, such as 0.1 to 30 μg/mL, 0.1 to 10 μg/mL, 1 to 5 μg/ml, such as 3 μg/mL. In some examples, the total dosages of the therapeutic protein, such as an apoptosis-modifying fusion protein, to be administered can range from 0.001 μg to 10,000 μg, from 0.01 μg to 1,000 μg or from 0.1 μg to 100 μg.

The disclosed compositions can also include one or more viscosity enhancing agents, for example which act to prevent backflow of the formulation when it is administered, for example by injection or via catheter. Such viscosity enhancing agents include, but are not limited to, biocompatible glycols and sucrose.

The pharmaceutical compositions provided herein can be formulated as a sterile injectable aqueous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in PBS/EDTA. In one example, the composition includes PBS/EDTA, for example 10 mM PBS/EDTA at pH 7.4. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides.

The disclosed compositions reduce the immunogenicity of therapeutic proteins, such as modified proaerolysin proteins, targeted cargo proteins, or apoptosis-modifying fusion proteins. It has been observed that addition of albumin, such as HSA, to a therapeutic protein composition (e.g., modified PA protein composition), significantly reduces that immunogenicity of the therapeutic protein (e.g., modified PA protein). For example, compositions that include both albumin and modified PA protein reduce immunogenicity (for example as evidenced by a reduction in the production of modified PA-specific antibodies) when administered to a human subject as compared to administration of the same compositions without albumin. Similar results are expected for apoptosis-modifying fusion proteins and targeted cargo proteins. Methods of measuring the antigenicity of a therapeutic protein can be assessed using methods known in the art. For example, the kinetics and magnitude of the antibody response to a modified PA, targeted cargo protein, or apoptosis-modifying fusion protein can be determined, for example, in immunocompetent mice, and can be used to facilitate the development of a dosing regimen that can be used in an immunocompetent human. Immunocompetent mice such as the strain C57-BL6 are administered intravenous doses of the therapeutic protein. Mice are sacrificed at varying intervals (e.g. following single dose, following multiple doses) and serum obtained. An ELISA-based assay can be used to detect the presence of the appropriate antibodies, such as anti-modified PA or apoptosis-modifying fusion protein antibodies.

In particular examples, use of the disclosed compositions reduces immunogenicity by at least 20%, at least 40%, at least 50%, at least 60%, at least 75%, at least 80%, at least 90%, at least 95%, at least 98% or at least 99% relative to the absence of the albumin. In some examples, immunogenicity is reduced by at least 2-fold, at least 3-fold, at least 4-fold, at least 5-fold, at least 6-fold, at least 7-fold, at least 8-fold, at least 9-fold, at least 10-fold or at least 20-fold, relative to the absence of the albumin. Methods of measuring immunogenicity are known in the art. For example, immunogenicity can be measured by monitoring the production of antibodies specific for the therapeutic protein, such as modified PA-specific or apoptosis-modifying fusion protein-specific antibodies (such as neutralizing antibodies) in subjects administered a therapeutically effective amount of therapeutic protein (such as modified PA protein, targeted cargo protein, or apoptosis-modifying fusion protein), such as a subject who receives at least one dose (such as at least 2, at least 3, or at least 5 doses, for example, 1, 2, 3, 4, 5, 6, 7, 8, 9, or 10 doses) of a therapeutically effective amount of therapeutic protein such as modified PA protein, targeted cargo protein, or apoptosis-modifying fusion protein. Exemplary methods are provided below.

The disclosure also provides a pharmaceutical pack or kit that includes one or more containers filled with one or more compositions provided herein. In some examples, the individual components (e.g., albumin and the therapeutic protein) are in separate containers in the kit. In one example, a container includes HSA (for example at a concentration of 0.5 to 25%, such as 1 to 5%, such as 2%) and a therapeutic protein such as modified PA protein (such as SEQ ID NO: 4) for example at a concentration of 0.5 to 10 μg/ml (for example 1 to 5 μg/ml, such as 3 μg/ml) in a total volume of 20 ml PBS/EDTA pH 7.4. In a specific example, a container includes 2% HSA and a modified PA protein (such as SEQ ID NO: 4) at 3 μg/ml in a total volume of 20 ml PBS/EDTA pH 7.4. In another specific example, a container includes 400,000 μg HSA and 60 μg of a modified PA protein (such as SEQ ID NO: 4) in a total volume of 20 mL 10 mM PBS/EDTA pH 7.4.

In another example, the kit includes a container containing HSA (for example at a concentration of 0.5 to 25%, such as 1 to 5%, such as 2%) and a therapeutic protein such as an apoptosis-modifying fusion protein (such as SEQ ID NO: 19, 21, or 25) for example at a concentration of 0.5 to 10 μg/ml (for example 1 to 5 μg/ml, such as 3 μg/ml) in a total volume of 10 ml PBS/EDTA pH 7.4. In a specific example, a container includes 2% HSA and an apoptosis-modifying fusion protein (such as SEQ ID NO: 19, 21, or 25) at 3 μg/ml in a total volume of 10 ml PBS/EDTA pH 7.4. In another specific example, a container includes 400,000 μg HSA and 60 μg an apoptosis-modifying fusion protein (such as SEQ ID NO: 19, 21, or 25) in a total volume of 20 mL 10 mM PBS/EDTA pH 7.4.

In yet another example, a kit includes containers wherein the HSA and the therapeutic protein such as a modified PA protein or apoptosis-modifying fusion protein are separated, and are then combined before administration to a subject. For example a kit can include a first container that includes HSA (for example at a concentration of 0.5 to 25%, such as 1 to 5%, such as 2%) and a second container that includes the modified PA protein or apoptosis-modifying fusion protein (for example 100 to 500 μg/ml, such as 300 μg/ml). In a specific example, the first container includes 2% HSA in PBS/EDTA pH 7.4 in a volume of 20 mls and the second container includes the modified PA protein (such as SEQ ID NO: 4) or an apoptosis-modifying fusion protein (such as SEQ ID NO: 19, 21 or 25) at 300 μg/ml in a total volume of 0.5 ml PBS/EDTA pH 7.4. In the clinic, 0.2 ml of the second vial are added to the first vial, to produce a 20 ml solution containing 3 μg/ml modified PA or apoptosis-modifying fusion protein.

Such a containers can be stored at 2−8° C. for at least 6 months, at −20° C. for at least 4 years, or 50° C. for at least 12 months.

The PBS/EDTA pH 7.4 can serve as a diluent for the compositions provided herein. In one example PBS/EDTA pH 7.4 is 10 mM PBS/EDTA pH 7.4, which can include: 20 mL water for injection (WFI), 10 mM sodium phosphate, 150 mM sodium chloride, and 1 mM EDTA. For example, 0.4 g of HSA can be added to 19.8 mL of this 10 mM PBS/EDTA pH 7.4 solution, and placed in a container, which can form part of a kit. In addition, 0.05 ml of a 3 mg/ml solution of a modified PA protein can be added to 0.45 mL of this 10 mM PBS/EDTA pH 7.4 solution, and placed in a container, which can form part of a kit.

### Modified Proaerolysin Molecules

The modified PA molecules useful in the disclosed compositions and methods include those PA proteins which have been modified to include a prostate-specific protease cleavage sequence. Exemplary modified PA molecules are provided in U.S. Pat. No. 7,282,476 and PCT Publication No. 2006/133553, both herein incorporated by reference. For example, the native furin site of PA (amino acids 427-432 of SEQ ID NO: 2) can be functionally deleted and replaced with a prostate-specific protease cleavage sequence. That is, the prostate-specific protease cleavage sequence functionally replaces the native furin cleavage site of PA. This replacement results in a proaerolysin variant that only becomes cytolytically active in the presence of enzymatically active protease. In one example, the entire furin site is deleted. For example, the furin cleavage site of PA (such as amino acids 427-432 of SEQ ID NO: 2) is deleted and a prostate-specific protease cleavage site (such as a PSA cleavage site), is inserted in its place. In other examples, the prostate-specific protease cleavage sequence is inserted into the furin site thereby inactivating the furin site such that the PA is no longer activated by furin, but instead is activated by a prostate-specific protease. In yet other examples, the furin cleavage site of PA is mutated (for example deleted or otherwise inactivated) and a prostate-specific protease cleavage site, such as a PSA cleavage site, inserted within, or added to the N- or C-terminus of the furin site.

Examples of prostate-specific protease cleavage sequences include, but are not limited to: a prostate-specific antigen (PSA) cleavage site, a prostate specific membrane antigen (PSMA) cleavage site, and a human glandular kallikrein 2 (hK2) cleavage site. PSA is a serine protease with the ability to recognize and hydrolyze specific peptide sequences. It is secreted by normal and malignant prostate cells in an enzymatically active form and becomes inactivated upon entering the circulation. Since neither blood nor normal tissue other than the prostate contains enzymatically active PSA, the proteolytic activity of PSA was used to activate protoxins at sites of prostate cancer. Examples of PSA cleavage sites include, but are not limited to, those shown in SEQ ID NOS: 5-15. In a particular example, the PSA cleavage site includes or consists of SEQ ID NO: 5.

Also disclosed are modified PA molecules in which the PA binding domain is modified such that it is functionally deleted. An exemplary binding domain sequence is shown in amino acids 1-83 of SEQ ID NO: 2 or 4. The binding domain can be functionally deleted using any method known in the art, for example by deletion of all or some of the amino acids of the binding domain, such as deletion of amino acids 1-83 of SEQ ID NO: 2 or 4, or deletion of one or more amino acids shown as amino acids 45-66 of SEQ ID NO: 2 or 4.

In yet other examples, the binding domain is functionally replaced with a prostate-tissue specific binding domain. For example, the binding domain can be deleted and a prostate-tissue specific binding domain inserted in its place. In one example, a prostate-tissue specific binding domain is linked to the N- or C-terminus of a modified PA protein that contains a functionally deleted native binding domain. The use of one or more prostate-tissue specific binding domains can increase targeting of modified PA proteins to prostate cells, such as prostate cancer cells and its metastases. Several prostate-tissue specific binding domains are known. Examples include, but are not limited to a luteinizing hormone releasing hormone (LHRH) sequence, such as those shown in SEQ ID NOS: 16 and 17, and antibodies that recognize PSA, prostate-specific membrane antigen (PSMA), human glandular kallikrein 2, or LHRH.

One or more prostate-tissue specific binding domains can be linked to one or more amino acids of a modified PA protein, but ideally, do not interfere significantly with the ability of the modified PA to be activated by a prostate-specific protease such as PSA, and the ability to form pores in cell membranes. For example, prostate tissue specific binding domains can be linked or inserted at an N- and/or C-terminus of a modified PA. In some examples, the native binding domain of PA is deleted (e.g., amino acids 1-83 of SEQ ID NO: 2 or 4), such that attachment or linking of a prostate tissue specific binding domain to the N-terminus results in attachment to amino acid 84 of SEQ ID NO: 2 or 4. In other examples, smaller deletions or point mutations are introduced into the native binding domain of PA, such that attachment or linking of a prostate tissue specific binding domain to the N-terminus results in attachment to amino acid 1 of SEQ ID NO: 2 or 4 (or whichever amino acid is N terminal following functional deletion of the native PA binding domain). In some examples, the N-terminal amino acid of PA is changed to a Cys or other amino acid to before attaching a prostate-tissue specific binding domain, to assist in linking the prostate-tissue specific binding domain to the modified PA protein.

Alternatively or in addition, one or more prostate tissue specific binding domains can be attached or linked to other amino acids of a modified PA protein, such as amino acid 215 or 300 of SEQ ID NO: 2 or 4. In some examples, a Cys amino acid replaces the native amino acid at that position. For example, the following changes can be made to SEQ ID NO: 2 or 4: Tyr215Cys or Ala300Cys. In one example, where the prostate tissue specific binding domain is an antibody, crosslinking can be used to attach antibodies to a modified PA, for example by reacting amino groups on the antibody with cysteine located in the modified PA (such as amino acids Cys19, Cys75, Cys159, and/or Cys164 of SEQ ID NO: 2).

In some examples, the modified PA proteins include a tag, such as a polyhistidine tag, for example a 6-His tag. Such tags can be present on the N- or C-terminus of the modified PA protein.

Particular modified PA proteins are shown in SEQ ID NO: 4 and 28, but one skilled in the art will appreciate that variants can be used, such as a sequence having at least 80%, at least 90%, at least 95%, at least 98%, or at least 99% sequence identity to SEQ ID NO: 4 and having a furin site replaced with a prostate-specific protease cleavage sequence.

### Apoptosis-Modifying Fusion Proteins

The apoptosis-modifying fusion proteins that can be formulated into a composition with albumin includes those proteins which include both an inactive toxin protein domain and an apoptosis regulating protein domain, wherein the inactive toxin protein domain targets the therapeutic protein to the cell but is not biologically active. Exemplary therapeutic proteins that can be formulated into such compositions are provided in U.S. Pat. No. 6,737,511 (herein incorporated by reference).

The inactive toxin protein domain can include a protein sequence from a toxin, such as an anthrax or diphtheria toxin, which is not toxic to a cell (such as a human cell), but still can target the protein to which it is linked to a cell. For example, the protein sequence from a toxin is one that does not lyse or otherwise kill a human cell when administered at a therapeutic dose, but can allow targeting to a desired cell. In some examples, the toxin is not Botulinum toxin. The physical interaction between a target cell and the inactive toxin protein domain can be examined by various methods. For example, ability of an apoptosis-modifying fusion protein to compete for binding to its target cell with either a native targeting domain or an antibody that recognizes the targeting domain binding site on the target cell can be measured. This allows the calculation of relative binding affinities through standard techniques.

In one example, the inactive toxin domain includes a portion of the diphtheria toxin (DT). Diphtheria toxin has three structurally and functionally distinct domains: (1) a cell surface receptor binding domain (DTR), (2) a translocation domain (DTT) that allows passage of the active domain across the cell membrane, and (3) the A (enzymatically active) chain that, upon delivery to a cell, ADP-ribosylates elongation factor 2 and thereby inactivates translation. In one example, the inactive toxin domain includes the receptor binding domain of DT (e.g., a receptor binding domain included within SEQ ID NO: 19).

In one example, the inactive toxin domain includes a portion of the anthrax toxin (AT). For example, the N-terminus of anthrax lethal factor (LF) can be used, such as amino acids 1-255 of mature anthrax LF (amino acids 1-255 of SEQ ID NO: 25), or a sequence having at least 90%, at least 95%, at least 98%, or at least 99% sequence identity to amino acids 1-255 of mature anthrax LF.

An apoptosis-modifying fusion protein can bind to a target cell, translocate across or otherwise integrates into the membrane(s) of the target cell, and modify an apoptotic response of the target cell. As such, any target cell in which it is desirous to modify (either inhibit or enhance) apoptosis is an appropriate target for a bispecific fusion protein. The choice of appropriate protein binding domain for incorporation into the disclosed apoptosis-modifying fusion protein will be dictated by the target cell or cell population chosen. Examples of targeting domains include, for instance, nontoxic cell binding domains or components of bacterial toxins (such as diphtheria toxin or anthrax toxin), growth factors (such as epidermal growth factor), monoclonal antibodies, cytokines, and so forth, as well as targeting competent variants and fragments thereof.

A translocation domain may be included in the fusion protein as a separate, third domain. This can be supplied from a third protein, unrelated to the cell-binding and apoptosis-modifying domains, or be a translocation domain of one of these proteins (e.g., the diphtheria toxin translocation (DTT) domain used in Bad-DTTR). The DTT domain contains several hydrophobic and amphipathic alpha helices and, after insertion into cell membranes, creates voltage dependent ion channels (Kagan et al., Proc Natl Acad Sci USA 78:4950-4954, 1981; Donovan et al., Proc Natl Acad Sci USA 78:172-176, 1981). Alternately, the translocation function can be provided through the use of a cell-binding domain or apoptosis-modifying domain that confers the additional functionality of membrane translocation or integration. This is true in Bcl-x_{L}-DTR, wherein Bcl-x_{L} provides both the apoptosis-modifying ability and translocation into the cell.

The apoptosis regulating protein domain can include a protein sequence that increases or decreases apoptosis, such as a Bcl-2 related protein or functional fragment or variant thereof (such amino acid residues 1-209 of Bcl-x_{L}). Since its discovery, several Bcl-2-related proteins (the Bcl-2 family of proteins) have been identified as being involved in regulation of apoptosis (White, Genes Dev. 10:1-15, 1996; Yang et al., Cell 80:285-291, 1995). One such is Bcl-x, which is expressed in two different forms, long (Bcl-x_{L}) and short (Bcl-xₛ) (Boise et al., Cell 74:597-608, 1993).

Bcl-x_{L} and certain other members of the Bcl-2 family are, like Bcl-2 itself, powerful inhibitors of cell death (the “anti-death” Bcl-2 family members). Other members of the Bcl-2 protein family, including Bcl-xₛ, Bad and Bax, are potent enhancers of apoptosis and therefore toxic to cells (“pro-death” Bcl-2 family members). It has been suggested that Bad binding to Bcl-x_{L} may promote cell death (Yang et al., Cell 80:285-291, 1995; Zha et al., J Biol. Chem 272:24101-24104, 1997) and that phosphorylation of Bad may prevent its binding to Bcl-x_{L}, thereby blocking cell death (Zha et al., J Biol. Chem. 272:24101-24104, 1997; Zha et al., Cell 87:619-628, 1996).

Where apoptosis is to be inhibited by the resultant fusion protein, anti-death members of the Bcl-2 protein family are appropriate sources for apoptosis-modifying domains. One such fusion protein is Bcl-x_{L}-DTR, which employs the long form of Bcl-x, Bcl-x_{L}, as the apoptosis-modifying domain. Alternately, where enhancement of apoptosis is desired, pro-death members of the Bcl-2 family of proteins will be appropriate. For instance, Bad-DTTR employs the pro-death protein Bad as its apoptosis-modifying domain.

In one example, the apoptosis-modifying domain is an apoptosis-enhancing domain. Such domains include the various pro-death members of the Bcl-2 family of proteins, for instance Bad, and variants or fragments thereof that enhance apoptosis in a target cell. A specific appropriate variant of the Bad protein has an amino acid other than serine at amino acid position 112 and/or position 136, to provide constitutively reduced phosphorylation. Thus, one specific embodiment is a functional apoptosis-enhancing fusion protein capable of binding a target cell, comprising the Bad protein and the diphtheria toxin translocation and receptor binding domains, functionally linked to each other. The Bad protein of this embodiment can also contain a mutation(s) at position 112 and/or 136 to change the serine residue to some other amino acid, to reduce phosphorylation of the protein. One such protein is Bad-DTTR; the nucleotide sequence of this protein is shown in SEQ ID NO: 20, and the corresponding amino acid sequence in SEQ ID NO: 21.

In one example, the apoptosis-modifying fusion protein is Bcl-x_{L}-DTR. This therapeutic fusion protein is encoded by the human Bcl-x_{L} gene from codon 1 through 233 and the diphtheria toxin gene from codon 384 through 535 (receptor binding domain, DTR), containing mutations in codons 508 and 525. Bcl-x_{L} is fused to the 5′ end of the DTR gene with a linker (GCG TAT TCT GCG GCC GCG, SEQ ID NO: 22) to encode for Ala Tyr Ser Ala Ala Ala (SEQ ID NO: 23) between the two peptide domains. The codon 508 of DTR was mutated to the wild-type form (Phe→Ser) and the first three nucleotides (CAT) of NdeI were deleted by double-stranded, site-directed mutagenesis. The Bcl-x_{L}-DTR sequence is provided in SEQ ID NOS: 18 and 19. However, one skilled in the art will appreciate that sequences having at least 90%, at least 95%, at least 98%, or at least 99% sequence identity to these sequences can be used.

In one example, the apoptosis-modifying fusion protein is Bad-DTTR. This fusion protein is encoded by the full-length mouse Bad gene with two Ser→Ala mutations at codons 112 and 136 (Schendel et al., Proc. Natl. Acad. Sci. USA 94:5113-5118, 1997), and the diphtheria toxin gene from codons 194 through 535 (translocation and receptor-binding domains, DTTR, without the catalytic domain). The Bad gene is fused to the 5′ end of DTTR gene. The Bad-DTTR sequence is provided in SEQ ID NOS: 20 and 21. However, one skilled in the art will appreciate that sequences having at least 90%, at least 95%, at least 98%, or at least 99% sequence identity to these sequences can be used.

In one example, the apoptosis-modifying fusion protein is LFₙ-Bcl-x_{L}. The N-terminal 255 amino acids of anthrax lethal factor (LFₙ) is fused to the 5′ end of the human Bcl-x_{L} gene. The LFₙ-Bcl-x_{L} sequence is provided in SEQ ID NOS: 24 and 25. However, one skilled in the art will appreciate that sequences having at least 90%, at least 95%, at least 98%, or at least 99% sequence identity to these sequences can be used.

### Other Targeted Cargo Proteins

In addition to the apoptosis-modifying fusion proteins discussed above, other types of targeted cargo proteins can be rendered less immunogenic using the compositions and methods provided herein.

Essentially any combination of cargo moiety and targeting moiety can be used. In this section exemplary combinations of targeting moieties and cargo moieties are provided. In all examples that targeting moiety can be an antibody that specifically binds to a target, such as a fully humanized antibody.

GMCSF can be used as a targeting moiety and linked to pro-apoptotic BCL-2 proteins, such as Bax, Bad, Bat, Bak, Bik, Bok, Bid, Bim, Bmf and Bok, as well as toxins such as aerolysin, proaerolysin or Pseudomonas exotoxin. Additionally, multiple cargo moieties can be linked to GMCSF or multiple GMCSF proteins can be linked to cargo moieties.

IL-4 (including IL-4 circularly permuted ligands and other IL-4 receptor binding proteins such as IL-13) is another targeting moiety that can be linked to BCL-2 family proteins, such as Bax, Bad, Bat, Bak, Bik, Bok, Bid, Bim, Bmf and Bok, or a toxin such as aerolysin, proaerolysin, Pseudomonas exotoxin, or combinations thereof. Additionally, multiple cargo moieties can be linked to IL-4 or multiple IL-4 proteins can be linked to cargo moieties. In one example, the targeted cargo protein is not PRX302.

IL-2 can be linked to BCL-2 family proteins, such as Bax, Bad, Bat, Bak, Bik, Bok, Bid, Bim, Bmf and Bok, or a toxin such as aerolysin, proaerolysin, Pseudomonas exotoxin or combinations thereof. Additionally, multiple cargo moieties can be linked to IL-2 or multiple IL-2 proteins can be linked to cargo moieties.

An antibody that binds to tenascin can be linked to BCL-2 family proteins, such as Bax, Bad, Bat, Bak, Bik, Bok, Bid, Bim, Bmf and Bok, or a toxin such as bouganin, aerolysin, proaerolysin, Pseudomonas exotoxin or combinations thereof. Additionally, multiple cargo moieties can be linked to the anti-tenascin antibody.

An antibody that binds to EpCAM can be linked to BCL-2 family proteins, such as Bax, Bad, Bat, Bak, Bik, Bok, Bid, Bim, Bmf and Bok, or a toxin such as bouganin, aerolysin, proaerolysin, Pseudomonas exotoxin or combinations thereof. Additionally, multiple cargo moieties can be linked to the anti-EpCAM antibody.

An antibody that binds to CD22 can be linked to BCL-2 family proteins, such as Bax, Bad, Bat, Bak, Bik, Bok, Bid, Bim, Bmf and Bok, or a toxin such as bouganin, aerolysin, proaerolysin, Pseudomonas exotoxin, or RNAse A or combinations thereof. Additionally, multiple cargo moieties can be linked to the anti-CD22 antibody.

An antibody that binds to mesothelin can be linked to BCL-2 family proteins, such as Bax, Bad, Bat, Bak, Bik, Bok, Bid, Bim, Bmf and Bok, or a toxin such as bouganin, aerolysin, proaerolysin, Pseudomonas exotoxin, or combinations thereof. Additionally, multiple cargo moieties can be linked to the anti-mesothelin antibody.

An antibody that binds to PSMA can be linked to BCL-2 family proteins, such as Bax, Bad, Bat, Bak, Bik, Bok, Bid, Bim, Bmf and Bok, a toxin such as bouganin, aerolysin, proaerolysin, Pseudomonas exotoxin, thapsigargin, a chemotherapeutic agent, or combinations thereof. Additionally, multiple cargo moieties can be linked to the anti-PSMA antibody.

EGF can be linked to BCL-2 family moieties, such as Bax, Bad, Bat, Bak, Bik, Bok, Bid, Bim, Bmf and Bok, or a toxin such as aerolysin, proaerolysin, Pseudomonas exotoxin or combinations thereof. Additionally, multiple cargo moieties can be linked to EGF or multiple EGF proteins can be linked to cargo moieties.

A circularly permuted ligand, for example a circularly permuted ligand derived from IL-4, IL-2, IL-3, IL-5, IL-10, IL-13, EGF, granulocyte colony stimulating factor (G-CSF) or granulocyte/macrophage colony stimulating factor (GMCSF) can be linked to a BCL-2 family protein, such as Bax, Bad, Bat, Bak, Bik, Bok, Bid, Bim, Bmf and Bok, bouganin, aerolysin, proaerolysin, Pseudomonas exotoxin or combinations thereof. Additionally, multiple cargo moieties can be linked to a circularly permuted ligand or multiple circularly permuted ligand proteins can be linked to cargo moieties.

Targeting moieties that are molecules that are natural ligands or derivatives of natural ligands to IL-4 receptors (IL-4R) on target cells can also be used. IL-4 can be used as the targeting moiety, and can be chemically or recombinantly linked to one or more of the cargo moieties described herein, such as. Examples of derivatives of natural ligands include the circularized cytokine ligands described in U.S. Pat. No. 6,011,002 to Pastan et al., herein incorporated by reference. In addition to IL-4 ligands, IL-13 can also be used as a ligand targeting moiety. In one embodiment, the IL-4 linked to Pseudomonas exotoxin is the PRX321 molecule shown in FIG. 6.

### Peptide Modifications

Although particular modified PA, albumin, apoptosis-modifying fusion protein sequences and other targeted cargo protein sequences are provided herein, one skilled in the art will appreciate that variations can be made to such proteins without significant adverse effects on the biological activity of the protein.

For example, the biological activity of the proteins disclosed herein lies not in the precise amino acid sequence, but rather in the three-dimensional structure inherent in the amino acid sequences encoded by the DNA sequences. It is possible to recreate the functional characteristics of any of these proteins or protein domains by recreating the three-dimensional structure, without necessarily recreating the exact amino acid sequence. This can be achieved by designing a nucleic acid sequence that encodes for the three-dimensional structure, but which differs, for instance by reason of the redundancy of the genetic code. Similarly, the DNA sequence may also be varied, while still producing a functional protein.

Variant proteins include proteins that differ in amino acid sequence from the sequences provided herein but that share structurally significant sequence homology with any of the provided proteins. Such variants may be produced by manipulating the nucleotide sequence of, for instance, SEQ ID NO: 3, 18, 20, or 24, using standard procedures, such as site-directed mutagenesis or PCR. The simplest modifications involve the substitution of one or more amino acids for amino acids having similar biochemical properties. These so-called conservative substitutions are likely to have minimal impact on the activity of the resultant protein, especially when made outside of the binding site or active site of the respective domain. For example, the regions or sub-domains of DTR that are essential to targeted cell binding are known in the art (see, Choe et al., Nature 357:216-222, 1992; Parker and Pattus, TIBS 18:391-395, 1993). Regions or sub-domains of Bcl-2 proteins responsible for apoptosis modification are reviewed in Adams and Cory, Science 281:1322-1326.

In one example, the disclosed proteins include no more than 50 conservative amino acid substitutions, such as 1 to 50, 5 to 40, 5 to 30, 2 to 20, 1 to 20, 1 to 10 or 1 to 5 such substitutions. More substantial changes in protein structure may be obtained by selecting amino acid substitutions that are less conservative than those listed above. Such changes include changing residues that differ more significantly in their effect on maintaining polypeptide backbone structure (e.g., sheet or helical conformation) near the substitution, charge or hydrophobicity of the molecule at the target site, or bulk of a specific side chain. The following substitutions are generally expected to produce the greatest changes in protein properties: (a) a hydrophilic residue (e.g., seryl or threonyl) is substituted for (or by) a hydrophobic residue (e.g., leucyl, isoleucyl, phenylalanyl, valyl or alanyl); (b) a cysteine or proline is substituted for (or by) any other residue; (c) a residue having an electropositive side chain (e.g., lysyl, arginyl, or histadyl) is substituted for (or by) an electronegative residue (e.g., glutamyl or aspartyl); or (d) a residue having a bulky side chain (e.g., phenylalanine) is substituted for (or by) one lacking a side chain (e.g., glycine).

Alternatively, the coding region may be altered by taking advantage of the degeneracy of the genetic code to alter the coding sequence such that, while the nucleotide sequence is substantially altered, it nevertheless encodes a protein having an amino acid sequence substantially similar to the disclosed fusion sequences. For example, the 57th amino acid residue of the Bcl-x_{L}-DTR protein is alanine. The nucleotide codon triplet GCC encodes this alanine residue. Because of the degeneracy of the genetic code, three other nucleotide codon triplets—(GCG, GCT and GCA)—also code for alanine. Thus, the nucleotide sequence of the disclosed Bcl-x_{L}-DTR encoding sequence could be changed at this position to any of these three alternative codons without affecting the amino acid composition or characteristics of the encoded protein.

### Activity of Apoptosis-Modifying Fusion Proteins

Because the apoptosis modifying fusion proteins provided herein are at least bi-functional, having one domain required for cell targeting and another for modification of apoptosis in the target cell, there are at least two activities for each fusion protein. These include the affinity of the fusion protein for a specific target cell, class of target cells, tissue type, etc., (the binding ability), and the ability of the targeted fusion to effect apoptosis in the targeted cell (the apoptosis-modifying ability). Various techniques can be used to measure each of these activities.

### Fusion Protein Affinity for Target Cells

Fusion protein affinity for the target cell, or to a specific cell surface protein, can be determined using various techniques known in the art, such as a competitive binding assay (Greenfield et al., Science 238:536-539, 1987). In a competitive binding assay, radiolabeled receptor binding protein, or a derivative or fragment thereof, is exposed to the target native cell in the presence of one or varying concentrations of cold fusion protein and other competitive proteins being assayed. The amount of bound, labeled binding protein can be measured through standard techniques to determine the relative cell-binding affinity of the fusion.

### Apoptosis Inhibition or Enhancement

Several in vitro systems are used to study the process of apoptosis. These include growth factor deprivation in culture, treatment of cells with staurosporine (a non-specific protein kinase inhibitor), application of γ-radiation, and infection by viruses. Apoptosis as stimulated by any signal can be measured. Detection of morphological indicia of apoptosis (e.g., membrane blebbing, chromatin condensation and fragmentation, and formation of apoptotic bodies) can provide qualitative information. More quantitative techniques include TUNEL staining, measurement of DNA laddering, measurement of known caspase substrate degradation (e.g., PARP; Taylor et al., J. Neurochem. 68:1598-605, 1997) and counting dying cells, which have become susceptible to dye uptake. Many companies (e.g., Trevigen, Gaithersburg Md.; and R&D Systems, Minneapolis Minn.) also supply kits useful for the measurement of apoptosis by various methods.

By way of example, the following techniques can be used to measure the modification of apoptosis caused in a target cell after it is contacted with an apoptosis-modifying fusion protein.

TUNEL staining: Terminal end-labeling of broken DNA fragments with labeled nucleotides; the reaction is catalyzed by terminal nucleotide transferase (TdT). Various kits are available for measurement of TUNEL staining, including the TdT in situ TUNEL-based Kit (R&D Systems, Minneapolis, Minn.).

Measurement of Caspase Activity: Poly-ADP ribose Polymerase (PARP) cleavage can be measured after treatment of the cells with various stimulators of apoptosis. HeLa cells are plated in growth media (e.g., EMEM containing 10% FBS at 2×10⁵ cells/ml) and treated with one or more concentrations of an apoptosis-modifying fusion protein provided herein. The appropriate concentration for each fusion protein will depend on various factors, including the fusion protein in question, target cell, and apoptosis stimulator employed, and may include about 0.5 μM to about 3 μM final. It may be beneficial to treat the target cells multiple times with the fusion protein, usually after a period of incubation ranging from one to several hours. For instance, cells can be exposed to the fusion protein a second time about fifteen hours after the original treatment.

Apoptosis is induced immediately the last treatment of the target cells with apoptosis modifying fusion protein. The method of application of the apoptosis stimulus, amount applied, appropriate incubation time with the inducer, etc., will be specific to the type of apoptosis induction used (e.g., staurosporine, γ-radiation, virions, caspase inhibitor, etc.). After an appropriate incubation period, cell lysates are prepared from the treated target cells, and aliquots loaded onto SDS-PAGE for analysis. The resultant gels can be examined using any of various well-known techniques, for instance by performing a Western analysis immunoblotted with anti-PARP polyclonal antibody (Boehringer Mannheim GmbH, Germany).

Known inhibitors of apoptotic pathways, for instance caspase inhibitors, can be used to compare the effectiveness of apoptosis-modifying fusion proteins of this invention. Appropriate inhibitors include viral caspase inhibitors like crmA and baculovirus p35, and peptide-type caspase inhibitors including zVAD-fmk, YVAD- and DEVD-type inhibitors. See Rubin, British Med. Bulle., 53:617-631, 1997.

### Methods of Reducing Antibody Response to a Therapeutic Protein

Provided herein are methods that can be used to decrease an antibody response to a therapeutic protein in a subject in need of treatment with the therapeutic protein. The method can include administering the therapeutic protein to the subject, wherein the therapeutic protein is present in a pharmaceutical composition that includes a sufficient amount of albumin to decrease the subject's antibody response to the therapeutic protein. A reduction in the antibody response to the therapeutic protein does not need to be 100% to be effective. For example, reductions of at least 10%, such as at least 20%, at least 30% at least 50%, at least 75%, at least 90%, at least 95% or more (for example relative to the antibody response to the therapeutic protein in the absence of albumin) can be achieved by the disclosed methods. In some examples the disclosed methods reduce an antibody response to the therapeutic protein by at least 2-fold, such as at least 3-fold, at least 4-fold, at least 5-fold, at least 6-fold, at least 8-fold, for example 2- to 10 fold (for example relative to the antibody response to the therapeutic protein in the absence of albumin). Methods of measuring antibody responses are known in the art, and particular examples are provided herein. In some examples, the disclosed methods also include measuring an antibody response to the therapeutic protein, for example at a time after the therapeutic protein is administered.

In some examples, the albumin is a human serum albumin (such as recombinant human serum albumin or a human serum albumin purified from human blood), and the subject is a human. The molar ratio of the amount of albumin to the amount of therapeutic protein can be at least 5:1, such as at least 10:1, at least 50:1, at least 1000:1, at least 5000:1, at least 10,000:1, at least 50,000:1, or at least 100,000:1, for example between 5:1 and 100,000:1 or between 50:1 and 5,000:1. In particular examples, the amount of albumin in the pharmaceutical composition is at least 0.01% by weight, such as at least 0.1%, at least 0.5%, at least 1%, at least 2%, at least 5%, at least 10%, at least 20%, or at least 25% by weight, for example between 0.01 and 25% by weight, between 0.2 and 5% by weight, or 2% by weight.

Exemplary therapeutic proteins are provided herein, including targeted cargo proteins, modified PA proteins, and apoptosis-modifying proteins. In one example the therapeutic protein is a targeted cargo protein that has a targeting moiety that specifically binds to a target displayed by a target cell, and a cargo moiety that exerts a biological effect on the target cell. For example, the targeted cargo protein can include one or more cargo moieties such as aerolysin, proaerolysin, bouganin, abrin, ricin, Pseudomonas exotoxin, cholera toxin, diphtheria toxin, tetanus toxin, neural thread protein and Bad. In some examples, the target is a receptor for IL-2, IL-4, IL-13, GM-CSF, EGF, proaerolysin toxin, diphtheria toxin, anthrax toxin and tetanus toxin. In some examples, the targeting moiety includes a ligand for a receptor for IL-2, IL-4, IL-13, GM-CSF, EGF, nicotinic acetylcholine, CD22 or GPI anchor protein. In some examples, the cargo moiety includes an aerolysin toxin, proaerolysin toxin, or Pseudomonas exotoxin. Specific exemplary targeted cargo proteins include Pseudomonas exotoxin linked to circularly permuted IL-4, IL-2 linked to aerolysin, IL-2 linked to proaerolysin, IL4 linked to BAD, GMCSF linked to BAD, EGF linked to proaerolysin, anti-EpCAM antibody linked to Pseudomonas exotoxin, anti-EpCAM antibody linked to bouganin, anti-mesothelin antibody linked to Pseudomonas exotoxin, anti-CD22 antibody linked to Pseudomonas exotoxin, anti-CD22 antibody linked to RNase A, and anti-PSMA antibody linked to thapsigargin. In specific examples, the therapeutic protein comprises Pseudomonas exotoxin linked to circularly permuted IL-4 and the molar ratio of the amount of the therapeutic protein to the amount of albumin is 5:1 to 5000:1, such as 50:1

In other specific examples, the therapeutic protein includes a modified proaerolysin (PA) protein. For example, a modified PA protein can include a prostate-specific protease cleavage site that replaces a proaerolysin furin cleavage site corresponding to amino acids 427-432. In a specific example, the modified proaerolysin protein includes a sequence having at least 90%, at least 95%, or 100% sequence identity to SEQ ID NO: 4. In a specific example, the modified PA protein is used to treat benign prostatic hyperplasia and the molar ratio of the modified PA protein to the albumin is at least 100:1, such as at least 500:1, at least 1000:1 or at least 10,000:1, for example, 500:1 to 50,000:1, such as 5000:1. In another specific example, the modified proaerolysin protein is used to treat benign prostate cancer, and the molar ratio of the modified proaerolysin protein to the albumin is at least 50:1, such as at least 100:1, at least 1000:1 or at least 5,000:1, for example, in the range of 50:1 to 5,000:1, such as 500:1.

In other specific examples, the therapeutic protein includes an apoptosis-modifying fusion protein comprising an inactive toxin protein domain, an apoptosis regulating protein domain, wherein the inactive toxin protein domain targets the fusion protein to the cell and is not biologically active. For example, the apoptosis regulating protein domain can include a Bcl-2 protein, such as a pro-apoptotic protein selected from Bcl-xs, Bax, Bad, Bak, DIVA, Bak, Bik, Bim, Bid and Egl-1, or an anti-apoptotic protein selected from Bcl-xL, Mcl-1, Ced-9 and A1. In one example, the inactive toxin protein domain comprises a domain derived from diphtheria toxin, tetanus toxin or anthrax toxin.

Exemplary modes of administration and dosages are provided throughout the specification. The disclosed therapeutic protein/albumin containing compositions can be administered more than once, such as every 6-12 months.

### Methods for Reducing Immunogenicity and Treating Prostate Cancer and BPH

Methods of using the disclosed compositions to reduce an immune response to a modified proaerolysin protein are provided. For example, the compositions provided herein that include albumin and a therapeutic protein (such as a modified PA protein) can be administered in a therapeutically effective amount to a subject in need of the therapeutic protein, thereby reducing an immune response in the subject to the therapeutic protein. Such compositions permit repeated administrations of the compositions, as the risk of producing neutralizing antibodies is reduced.

Thus in some examples the method includes measuring an antibody response to the therapeutic protein. For example, antibodies specific for the therapeutic protein can be measured in a sample obtained from the subject (such as a blood sample or fraction thereof such as serum or plasma). In some examples, such measurements are made before administration of a composition containing albumin and a therapeutic protein, after administration of such composition, or both. In some examples, the anti-therapeutic antibodies produced by the subject are quantified. The presence of the albumin reduces the production of antibodies to the therapeutic protein in the subject, for example relative to administration of the therapeutic protein without albumin. In some examples, a reduction of at least 10%, at least 20%, at least 50%, at least 75%, or at least 90% is observed.

The disclosed compositions can be administered as a single modality therapy or used in combination with other therapies, such as radiation therapy and/or androgen ablative therapies (such as LHRH receptor agonists/antagonists, antiandrogens, estrogens, adrenal steroid synthesis inhibitors ketoconazole and aminoglutethimide).

In one example, the disclosed compositions containing albumin and a modified PA are used in combination with one or more additional treatments for BPH. The additional treatments for BPH include administration of drugs such as α-1-adrenoreceptor antagonists and 5-α reductase inhibitors, phytotherapies, surgical procedures, and minimally invasive techniques. Examples of α-1-adrenoreceptor antagonists are alfuzosin/prazosin, tamsulosin, terazosin, and doxazosin. Examples of 5-α reductase inhibitors are finasteride and dutasteride. Examples of phytotherapies include Saw palmetto berry/dwarf palm (Serenoa repens), African plum bark (Pygeum africanum), South African star grass/beta-sitosterol (Hipoxis rooperi), Purple cone flower (Echinacea purpurea), Pumpkin seeds (Cucurbita pepo), Rye (Secale cereale), and Stinging nettle (Urtica dioica). Examples of surgical procedures are transurethral resection of the prostate (TURP), transurethral needle ablation (TUNA), transurethral incision of the prostate (TUIP), transurethral microwave thermotherapy (TUMT), laser prostatectomy, balloon dilation, electrical vaporization and open prostatectomy.

In addition, administration of the disclosed compositions can be alone, or in combination with a pharmaceutically acceptable carrier (such as a pharmaceutically and physiologically acceptable fluid), and/or in combination with other therapeutic compounds, such as other anti-tumor agents, immunosuppressants (such as Rituximab, steroids), and cytokines (such as GM-CSF). Examples of pharmaceutically effective carriers are discussed above, and include, but are not limited to water, physiological saline, balanced salt solutions (such as PBS/EDTA), aqueous dextrose, sesame oil, glycerol, ethanol, combinations thereof, or the like, as a vehicle. The carrier and composition can be sterile, and the formulation suits the mode of administration.

Subjects in need of such proteins can include human subjects having prostate cancer or BPH, wherein the modified PA protein treats the prostate cancer or BPH. For example, the disclosed compositions can be used as initial treatment for localized prostate cancer either alone, or in combination with radiation (external beam or brachytherapy) and/or androgen ablation therapy. The disclosed compositions can also be administered to patients who have failed radiation therapy and are suspected to only have a local recurrence of prostate cancer within the prostate gland. The disclosed compositions can also be given to patients with localized and metastatic prostate cancer to treat the localized cancer directly and to treat the metastatic cancer via stimulation of a systemic anti-tumor immune response. In other examples, the disclosed compositions can be used as initial treatment for BPH, or given to BPH patients who have received other therapies (such as alpha blockers, for example Flomax™) that did not successfully treat the BPH.

Thus, in some examples the methods provided herein treat prostate cancer or BPH in a subject. In some examples, the method includes selecting a subject that will benefit from the disclosed compositions, such as selecting a subject having prostate cancer (such as a localized or metastatic prostate tumor) or BPH. In some examples, the subject is one who has received other therapies, but those other therapies have not provided a desired therapeutic response.

In one example, administration of the disclosed compositions to a subject having prostate cancer or BPH decreases the immunogenicity of the modified PA in the composition. For example, the disclosed methods can reduce the production of modified PA-specific antibodies (such as a neutralizing antibody, for example one that significantly reduces the ability of the modified PA to lyse or kill prostate cancer cells) by at least 10%, for example by at least 20%, at least 40%, at least 50%, at least 80%, at least 90%, such at least 2-fold, at least 5-fold, at least 10-fold, or at least 20-fold, relative to the absence of albumin in the composition. Methods of monitoring or measuring antibodies present in a subject, such as in a serum sample from the subject, are routine, and exemplary ELISA methods are provided herein.

In one example, administration of the disclosed compositions to a subject having prostate cancer decreases the volume of a prostate tumor, slows the growth of a prostate tumor, decreases or slows metastasis of the tumor, or combinations thereof. For example, the disclosed methods can reduce prostate tumor cell volume and/or a metastatic tumor cell volume, such as by at least 10%, for example by at least 20%, at least 40%, at least 50%, at least 80%, at least 90%, or more, relative to the absence of administration of the composition. In addition, the disclosed methods can result in a decrease in the symptoms associated with a prostate tumor and/or a metastatic prostate tumor. In one example, administration of the disclosed compositions slows the growth of a prostate tumor, such as by at least 10%, for example by at least 20%, at least 40%, at least 50%, at least 80%, at least 90%, or more, relative to the absence of administration of the composition. Methods of monitoring prostate tumor volume/size/metastasis are routine in the art.

The compositions containing albumin and a modified PA protein are capable of selectively killing normal prostate cells in vivo, and are capable of decreasing the weight or volume of normal prostate gland in vivo. By selective killing of normal prostate cells relative to cells from other normal tissues is meant that the modified PA proteins are capable of killing normal prostate cells more effectively than other types of normal cells such as, for example, lung, spleen, or blood cells. Modified PA proteins are capable of decreasing the size of the prostate gland, or attenuating further growth of the prostate gland and thus are suitable for the treatment of BPH. In one example, administration of the disclosed compositions to a subject having BPH decreases the volume, weight, or size of the prostate or reduces one or more other negative side effects of BPH. In one example, administration of the disclosed compositions reduces the size, weight, or volume of a prostate in a subject with BPH by at least 10%, for example by at least 20%, at least 40%, at least 50%, at least 80%, at least 90%, or more, relative to the absence of administration of the composition. Methods of monitoring prostate size, weight, and volume are routine in the art (for example, planimetry, prolate ellipse volume calculation (HWL), and an ellipsoid volume measurement technique). Prostate size can also be measured directly, for example by digital rectal examination, or rectal ultrasound or cytoscopy, or indirectly, for example, by measuring changes in the levels of blood PSA or changes in the proportions of free and total PSA in the blood. In one example, administration of the disclosed compositions to a subject having BPH attenuates further growth of the prostate gland, which can be measured using routine methods, such as by a reduction in the rate of increase in the volume or the rate of increase of blood PSA or reduction in symptoms of BPH as described above. Decreasing the size of the prostate gland refers to a decrease in the weight or volume of a prostate gland, and attenuating of further growth of the prostate gland refers to the situation where there is minimal or no increase in the weight or volume of a prostate gland in a subject subsequent to administration of the therapeutic composition.

In another embodiment, treatment of BPH refers to the decrease in the degree of severity of one or more symptoms of BPH. Symptoms of BPH include changes or problems with urination, such as a hesitant, interrupted or weak stream, urgency and leaking or dribbling, or more frequent urination, especially at night. These symptoms are also known as lower urinary tract symptoms (LUTS). LUTS can be measured as known in the art using the American Urological Association (AUA) Symptom Index, the Madsen-Iversen Scoring System, or the Boyarsky System.

Disclosure of certain specific examples is not meant to exclude other embodiments. In addition, any treatments described herein are not necessarily exclusive of other treatment, but can be combined with other bioactive agents or treatment modalities.

### Administration of Compositions

The disclosed compositions can be administered locally or systemically using any method known in the art, for example to subjects having localized or metastatic prostate cancer or having BPH. In one example, the disclosed compositions are administered by parenteral means, including direct injection into a prostate (intraprostatically). For example, an administration approach similar to the multiple injection approach of brachytherapy can be used, in which multiple aliquots of the disclosed compositions, adapted as compositions or formulations and in the appropriate dosage form, can be injected using a needle through the perineum. Alternatively, the disclosed compositions are administered by direct injection or infusion into a prostate tumor (intratumorally).

Alternative methods of administration of the disclosed compositions will be evident to one of ordinary skill in the art. Such methods may include for example, the use of catheters or implantable pumps to provide continuous infusion over a period of several hours to several days into the subject in need of treatment. It is anticipated that active agents will be administered by the routes that are currently in use for their administration in clinical settings. Localized injection and subsequent lysis of prostate cells (such as prostate cancer cells) within the prostate gland can produce an immunostimulatory effect leading to a decrease or elimination of micrometastatic disease in treated subjects. In this way, systemic disease is treated or reduced through a minimally toxic, locally applied therapy.

In a specific example, the disclosed composition is injected into the prostate gland of patients according to a predefined template similar to that used to administer intraprostatic brachytherapy. The techniques and equipment required for intraprostatic administration are also similar to those used for brachytherapy and have been previously described (Deweese et al., Cancer Res. 61:7464-72, 2001). In another example, the intraprostatic injection of compositions disclosed herein are assisted with transrectal ultrasound according to a procedure similar to that used to perform transrectal ultrasound-assisted biopsies. Patients can receive multiple injections (20-80) at predefined sites to encompass the entire prostate gland. The total dose of administered modified PA will range from about 0.001-1.0 mg, and not more than 10 mg total. Patients are treated as in patients and monitored in the hospital for 48 hours post injection. Subsequently, patients will be examined weekly for signs of toxicity. MRI of the prostate can be used to monitor direct treatment effect on prostatic size. Immune response to intraprostatic modified PA can be monitored as previously described (Simons et al. Cancer Res. 59:5160-8, 1999).

In one example, a therapeutically effective amount of intraprostatically administered modified PA can vary from 0.01 to 50 μg per gram prostate weight, 0.02 to 40 μg modified PA per gram prostate weight, 0.02 to 35 μg modified PA per gram prostate weight, 0.03 to 25 μg modified PA per gram prostate weight, 0.04 to 20 μg modified PA per gram prostate weight, or 0.04 to 10 μg modified PA per gram prostate weight. In yet another example, a therapeutically effective amount of intravenously administered modified PA can vary from 1 mg to about 10 mg of modified PA, 1 mg to 5 mg, 1 mg to 3 mg, or 2.8 mg modified PA. In one embodiment, an effective intraprostatic dose of a modified PA for a 70 kg human is from 10 mg to 100 mg of modified PA, such as 10 mg to 50 mg, 10 mg to 30 mg, or 28 mg modified PA for a 70 kg human.

In one embodiment, the composition containing albumin and a modified PA is administered intraprostatically at a dose range from 0.01 μg/g prostate to 50 μg/g prostate, such as 0.02 μg/g prostate to 40 μg/g prostate, 0.02 μg/g prostate to 35 μg/g prostate, 0.03 μg/g prostate to 25 μg/g prostate, 0.04 μg/g prostate to 20 μg/g prostate, 0.04 μg/g prostate to 10 μg/g prostate, 0.1 μg/g prostate to 5 μg/g prostate, 0.2 μg/g prostate to 3 μg/g prostate, or 0.5 μg/g prostate to 2 μg/g prostate.

Exemplary dosages of the HSA to a subject for a single treatment may range from 4000 μg per dose to 4,000,000 μg/dose, such as 10,000 μg/dose to 2,000,000 μg/dose, 40,000 μg/dose to 1,000,000 μg/dose, 80,000 μg/dose to 800,000 μg/dose, 100,000 μg/dose to 600,000 μg/dose, for example about 400,000 μg/dose, for example if administered intraprostatically or intratumorally.

Dosages of the active agents are determined in accordance with current clinical protocols for the active agent being used. It is anticipated that the therapeutic dosage of the modified PA protein when used in combination with albumin may be increased from what would otherwise be determined to be the optimal level for the modified PA protein in the absence of the albumin as the albumin reduces the immunogenicity of the modified PA protein.

Treatments with disclosed compositions can be completed in a single day, or may be done repeatedly on multiple days with the same or a different dosage. Repeated treatments may be done on the same day, on successive days, or every 1-3 days, every 3-7 days, every 1-2 weeks, every 2-4 weeks, every 1-2 months, every 3-6 months, every 6 to 12 months, every 1-2 years, every 2-5 years, or at even longer intervals.

### Methods for Reducing Immunogenicity and Modulating Apoptosis

Methods of using the disclosed compositions to reduce an immune response to a therapeutic protein, such as an apoptosis-modifying fusion protein are provided. For example, the compositions provided herein that include albumin and an apoptosis-modifying fusion protein can be administered in a therapeutically effective amount to a subject in need of modified apoptosis (such as increased or decreased apoptosis), thereby reducing an immune response in the subject to the apoptosis-modifying fusion protein. Such compositions permit repeated administrations of the compositions, as the risk of producing neutralizing antibodies is reduced.

In addition to its involvement in neuronal and lymphoid system development and overall cell population homeostasis, apoptosis also plays a substantial role in cell death that occurs in conjunction with various disease and injury conditions. For instance, apoptosis is involved in the damage caused by neurodegenerative disorders, including Alzheimer's disease (Barinaga, Science 281:1303-1304), Huntington's disease, and spinal-muscular atrophy. There is also a substantial apoptotic component to the neuronal damage caused during stroke episodes (reviewed in Rubin, British Med. Bulle., 53(3):617-631, 1997; and Barinaga, Science 281:1302-1303), and transient ischemic neuronal injury, as in spinal cord injury. Thus, the compositions provided herein can be used to treat subjects having such disorders.

Methods for modifying apoptosis in a target cell are also encompassed, wherein a sufficient amount of a composition containing an apoptosis-modulating fusion protein and albumin can be used to modify apoptosis in the target cell by contacting the composition with a target cell. Modification of apoptosis can be by either inhibition or enhancement of an apoptotic response of the target cell. The apoptosis-modulating fusion protein can be administered to the target cell in the form of a pharmaceutical composition, and can further be administered with various medical or therapeutic agents, and/or additional apoptosis modifying substances. Such agents may include, for instance, chemotherapeutic, anti-inflammatory, anti-viral, and antibiotic agents.

The disclosed compositions can be administered as a single modality therapy or used in combination with other therapies, for instance chemotherapeutic, anti-inflammatory, or anti-viral or antibiotic therapies. For instance, an apoptosis-enhancing fusion protein such as Bad-DTTR may be combined with or used in association with other chemotherapeutic or chemopreventive agents for providing therapy against neoplasms or other hyper-proliferative cellular growth conditions. Various such anti-cancer agents are well known to those of ordinary skill in the art.

In addition, administration of the disclosed compositions can be alone, or in combination with a pharmaceutically acceptable carrier (such as a pharmaceutically and physiologically acceptable fluid), and/or in combination with other therapeutic compounds, such as other chemotherapeutic, anti-inflammatory, or anti-viral or antibiotic compositions. Examples of pharmaceutically effective carriers are discussed above, and include, but are not limited to water, physiological saline, balanced salt solutions (such as PBS/EDTA), aqueous dextrose, sesame oil, glycerol, ethanol, combinations thereof, or the like, as a vehicle. The carrier and composition can be sterile, and the formulation suits the mode of administration.

Subjects in need of such proteins can include human subjects having a disorder associated with undesired or desired apoptosis, such as neurodegenerative diseases, transient ischemic injuries, and unregulated cell growth (as may for instance be found in tumors and various cancers). Thus, in some examples the methods provided herein treat a neurodegenerative disease, transient ischemic injury, or uncontrolled growth (such as a tumor) in a subject. In some examples, the method includes selecting a subject that will benefit from the disclosed compositions, such as selecting a subject having a neurodegenerative disease, transient ischemic injury, or uncontrolled growth (such as a tumor). In some examples, the subject is one who has received other therapies, but those other therapies have not provided a desired therapeutic response.

In one example, administration of the disclosed compositions to a subject having a neurodegenerative disease, transient ischemic injury, or uncontrolled growth (such as a tumor) decreases the immunogenicity of the apoptosis-modifying fusion protein in the composition. For example, the disclosed methods can reduce the production of apoptosis-modifying fusion protein-specific antibodies (such as a neutralizing antibody, for example one that significantly reduces the ability of the apoptosis-modifying fusion protein to increase or decrease apoptosis of a desired cell) by at least 10%, for example by at least 20%, at least 40%, at least 50%, at least 80%, at least 90%, such at least 2-fold, at least 5-fold, at least 10-fold, or at least 20-fold, relative to the absence of albumin in the composition. Methods of monitoring or measuring antibodies present in a subject, such as in a serum sample from the subject, are routine, and exemplary ELISA methods are provided herein.

In one example, administration of the disclosed compositions containing an apoptosis-modifying fusion protein and albumin to a subject having a neurodegenerative disease, transient ischemic injury, or uncontrolled growth (such as a tumor) increases or decreases apoptosis of a target cell. For example, if the subject has a tumor, an increase in apoptosis of the tumor cells can be achieved. If the subject has a neurodegenerative disease, a decrease in apoptosis of neural cells can be achieved. For example, Bcl-x_{L}-DTR, LFₙ-Bcl-x_{L}, or related fusion proteins can be used to inhibit apoptosis in a target cell by contacting the target cell with an amount of this protein sufficient to inhibit apoptosis. Alternatively, Bad-DTTR or related fusion proteins can be used to enhance apoptosis in a target cell by contacting the target cell with an amount of this protein sufficient to enhance apoptosis. For example, the disclosed methods can increase or decrease apoptosis by at least 10%, for example by at least 20%, at least 40%, at least 50%, at least 80%, at least 90%, or more, relative to the absence of administration of the composition.

A specific aspect disclosed herein is the method of reducing apoptosis in a subject after transient ischemic neuronal injury, for instance a spinal cord injury, comprising administering to the subject a therapeutically effective amount of a composition containing albumin and an apoptosis-inhibiting protein. Examples of such fusion proteins include Bcl-x_{L}-DTR and LFₙ-Bcl-x_{L}. These proteins can be administered in the form of a pharmaceutical composition, and can be co-administered with various medical or therapeutic agents, and/or additional apoptosis modifying substances.

The apoptosis-modifying fusion proteins and albumin-containing compositions can be administered to humans, or other animals on whose cells they are effective, in various manners such as, intravenously, intramuscularly, intraperitoneally, intradermally, intrathecally, and subcutaneously. Administration of apoptosis-modifying fusion protein composition is indicated for patients with a neurodegenerative disease, suffering from stroke episodes or transient ischemic injury, or experiencing uncontrolled or unwanted cell growth, such as malignancies or neoplasms. More generally, treatment is appropriate for any condition in which it would be beneficial to alter (either inhibit or enhance) an apoptotic response of a subject's target cells. The particular mode of administration and the dosage regimen will be selected by the attending clinician, taking into account the particulars of the case (e.g., the patient, the disease, and the disease-state involved). By way of example, when apoptosis is being generally inhibited over the short term, for instance after transient ischemic neuronal injury, it may be advantageous to administer relatively large doses of fusion protein repeatedly for a few days. In contrast, if apoptosis is being enhanced in specific cell types, for instance in hyper-proliferative cells, it may be of greater benefit to apply a relatively small dose of fusion protein repeatedly, e.g., daily, weekly, or monthly, over a much longer period of treatment.

The pharmaceutical compositions that comprise apoptosis modifying fusion protein can be formulated in unit dosage form, suitable for individual administration of precise dosages. One possible unit dosage contains approximately 100 μg of protein. The amount of active compound administered will be dependent on the subject being treated, the severity of the affliction, and the manner of administration, and is best left to the judgment of the prescribing clinician. Ideally, a sufficient amount of the protein is administered to achieve tissue a concentration at the site of action that is at least as great as in vitro concentrations that have been shown to be effective.

### EXAMPLES

PRX302 (SEQ ID NO: 4 and 28) is a bacterial-derived protein and therefore is expected to be immunogenic. The examples below describe studies where antibody production was evaluated in the rat, cynomolgus monkey, and in human phase I and II studies in patients with prostate cancer and BPH using the “old” (no HSA) and “new” (includes HSA) formulations of PRX302.

### Example 1

### PRX302 Pharmaceutical Composition and Injection Kit

The drug product consists of PRX302 diluted using a formulation buffer containing sodium dihydrogen phosphate, disodium hydrogen phosphate, sodium chloride, disodium ethylenediaminetetraacetic acid, and water, pH 7.4), to produce the drug product at a target concentration of 300 μg/mL.

TABLE 4
Composition of Drug Product (per 2 mL vial with 0.55 g fill weight)

| | | | Quality |
|---|---|---|---|
| Component | Function | Quantity | Standard |
| PRX302 Drug Substance | Active | 0.165 mg | Company |
| 380 μg/mL - formulated | Ingredient | | Standard |
| in 0.7 mM sodium dihydrogen | | | (GMP) |
| phosphate, 9.2 mM disodium | | | |
| hydrogen phosphate, | | | |
| 150 mM sodium chloride, | | | |
| and 1 mM ethylenediamine | | | |
| tetraacetic acid, at a pH of 7.4 | | | |
| Formulation Buffer | Diluent | 544.835 mg | USP |

Abbreviations:
EDTA, Ethylenediaminetetraacetic Acid;
GMP, Good Manufacturing Practice;
USP, United States Pharmacopeia

TABLE 5
Composition of Formulation Buffer (per 2000 mL)

| | | | | Quality |
|---|---|---|---|---|
| Component | Function | Quantity | | Standard |
| Sodium dihydrogen | Buffer | 0.194 | g | USP |
| phosphate, monohydrate | | | | |
| (NaH₂PO₄•H₂0), 0.7 mM | | | | |
| Disodium hydrogen | Buffer | 6.588 | g | Ph. Eur./ |
| phosphate, dodecahydrate | | | | USP |
| (Na₂HPO₄•12H₂0), 18 mM | | | | |
| Sodium chloride (NaCl), | Osmolality | 17.534 | g | Ph. Eur./ |
| 150 mM | | | | USP |
| Disodium | Stabilizer | 0.744 | g | Ph. Eur. |
| ethylenediaminetetraacetic | | | | |
| acid, dehydrate | | | | |
| (Na₂EDTA•2H₂O), 1 mM | | | | |
| Water For Injections | Solvent | q.s. to 2000 | mL | Ph. Eur. |
| (WFI) | | | | |

Abbreviations:
Ph. Eur., European Pharmacopoeia;
USP, United States Pharmacopeia;
WFI, Water For Injections;
q.s., quantities sufficient

PRX302 is prepared for injection by dilution of an appropriate amount of the 300 μg/mL PRX302 drug product solution with an appropriate quantity of the diluents solution described below to produce the drug product at the target concentration, such as 0.75 μg/mL, 1.5 μg/mL, 3.0 μg/mL, or 6.0 μg/mL.

TABLE 6
Composition of Diluent - Per 20 mL Vial

| | Quantity (mg) Per | | Con- | | Quality |
|---|---|---|---|---|---|
| Component | 20 mL Vial | | centration | | Standard |
| Human Serum Albumin | 1700.886 | mg | 2% | (w/v) | USP |
| (HSA), 25% | | | | | |
| solution | | | | | |
| Sodium dihydrogen | 1.787 | mg | .6 | mM | USP |
| phosphate, monohydrate | | | | | |
| (NaH₂PO₄•H₂0) | | | | | |
| Disodium hydrogen | 60.700 | mg | 8.5 | mM | Ph. Eur./ |
| phosphate, dodecahydrate | | | | | USP |
| (Na₂HPO₄•12H₂0) | | | | | |
| Sodium chloride | 161.554 | mg | 138 | mM | Ph. Eur./ |
| (NaCl) | | | | | USP |
| Disodium | 6.840 | mg | 0.92 | mM | Ph. Eur. |
| ethylenediaminetetraacetic | | | | | |
| acid, dihydrate | | | | | |
| (Na₂EDTA•2H₂O) | | | | | |
| Water For Injections | q.s. to 20 | mL | | | Ph. Eur. |
| (WFI) | | | | | |

Abbreviations:
Ph. Eur., European Pharmacopoeia;
USP, United States Pharmacopeia;
WFI, Water For Injections;
q.s., quantities sufficient

### Example 2

### Immunogenicity in the Absence of Albumin

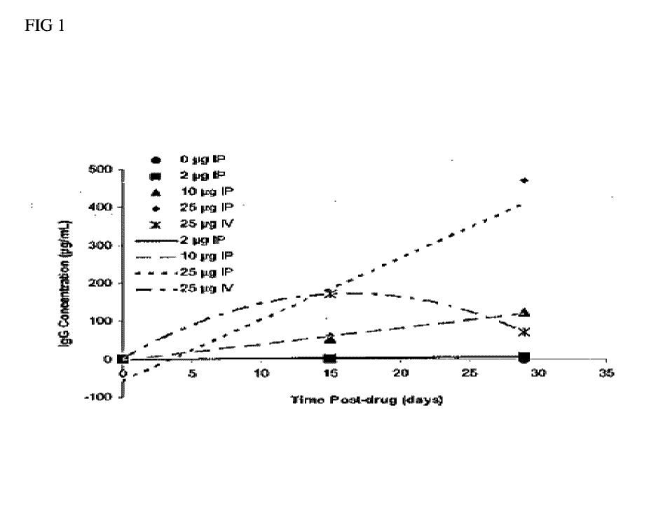

Mice were administered a single dose of PRX302 (0 to 25 μg, see FIG. 1) intraprostatically (IP) or intravenously (IV) in PBS-EDTA. Monkeys were administered a single dose of PRX302 (0 to 9.45 μg/kg of body weight) intraprostatically in the absence of added HSA. Subsequently, serum samples were analyzed for the presence of antibodies specific for the modified PA (PRX302). The validated Enzyme Linked Immunosorbent Assay (ELISA) based detection method is specific for only anti-PRX302 antibodies. This assay involved a 96-well plate previously coated with PRX302 solution. Anti-PRX302 antibodies in serum test samples are captured by PRX302 on the plate. The blocking buffer used was 1% bovine serum albumin (BSA) which specifically prevents non-specific binding. Peroxidase coated anti-human IgG was added for competitive binding for pre-coated PRX302 with the serum test samples already added to the wells. O-phenylenediamine (OPD 2HCl) peroxidase substrate was added to develop the colour. The colour reaction was stopped by adding a stopping reagent and the absorbance was measured at 492 nm using ELISA reader.

As shown in FIG. 1, in rats, antibody response to the modified PA protein in the absence of albumin was dose-dependent and increased with time following intraprostatic PRX302 administration. Following intravenous treatment, response peaked around 15 days post-treatment and started to taper off around Day 29.

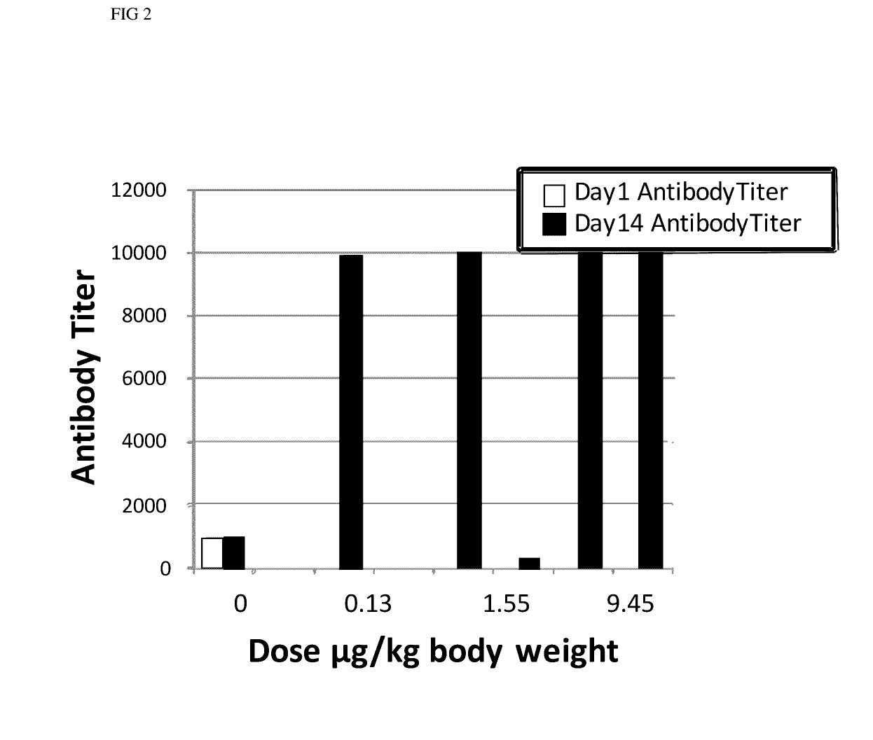

A second study was performed in cynomolugus monkeys. These animals received an intraprostatic injection at a dose of 0, 0.13, 1.55 or 9.45 μg/kg of body weight of PRX302 in a 25 μL volume of PBS-EDTA (these doses are equivalent to 0, 1, 5 and 25 μg/g prostate). Antibody titer was determined on the day of dosing (Day 1) and at Day 14 post-dosing (FIG. 2). A baseline titer of antibody of 1:1000 was observed in one animal receiving saline injection at Day 1 and Day 14. One of two animals receiving 0.13 or 1.55 μg/kg of body weight (1 or 5 μg/g prostate) developed high titer antibody at Day 14, while both animals receiving 9.45 μg/kg of body weight (25 μg/g prostate) had high titer antibodies. Overall 67% ( 4/6 animals) developed high titer antibody. The current maximum human dose of PRX302 in BPH treatment is approximately 0.75 μg/kg of body weight, and for prostate cancer treatment is approximately 4.0 μg/kg of body weight.

Thus administration of a modified PA protein results in an immune response, as indicated by the production of anti-modified PA antibodies.

### Example 3

### Anti-PRX302 Antibodies are Neutralizing

The ability of PRX302 antibody to neutralize the toxicity of PRX302 was studied in vitro using a PSA-producing human prostate cancer cell line LNCaP.

To determine if the PRX302 antibodies were neutralizing, the assay employed the human prostate cancer cell line LNCaP. This prostate cancer cell line produces PSA. To evaluate serum for neutralizing antibody, LNCaP cells were plated and exposed to PRX302 at a concentration of 3.6 nM in the absence or presence of serum samples from animals or patients administered PRX302. As a positive (neutralizing) control, anti-PRX302 antibody is added to 10% human serum. Other controls include serum alone and serum plus PRX302. Test serum samples are then added at various dilutions in combination with PRX302. Cells are exposed under these conditions for 72 hrs and then cell viability determined using an MTT based 96 well cell proliferation assay (Promega). The assay is designed such that the dose of PRX302 in the absence of neutralizing antibody kills >75% of the cells. Samples are considered positive if the sample produces a ≧10% reduction in number of cells killed vs. controls.

The first human clinical trial with PRX302 was a Phase I intraprostatic study in men with evidence of recurrence after radiation therapy for localized prostate cancer. In this study, 24 men were treated in 7 cohorts with increasing concentration of PRX302 Patients were injected intraprostatically with 100 μL per gram of prostate tissue of a solution containing 0.3, 0.9, 3.0, 6.0, 12.0, 21.0, or 30.0 μg/mL of PRX302 in PBS-EDTA. Patients received total doses of PRX302 ranging from 0.7 μg to 89 μg. To prevent loss of PRX302 resulting from the protein sticking to the walls of the glass vials used to dilute frozen stock solutions of PRX302 to the appropriate concentration for injection, the glass vials were pre-coated with a 2% solution of HSA, which was then discarded before PRX302 was introduced into the vials. Injections were done transperineally, under transrectal sonograpnic guidance using a modified brachytherapy technique.

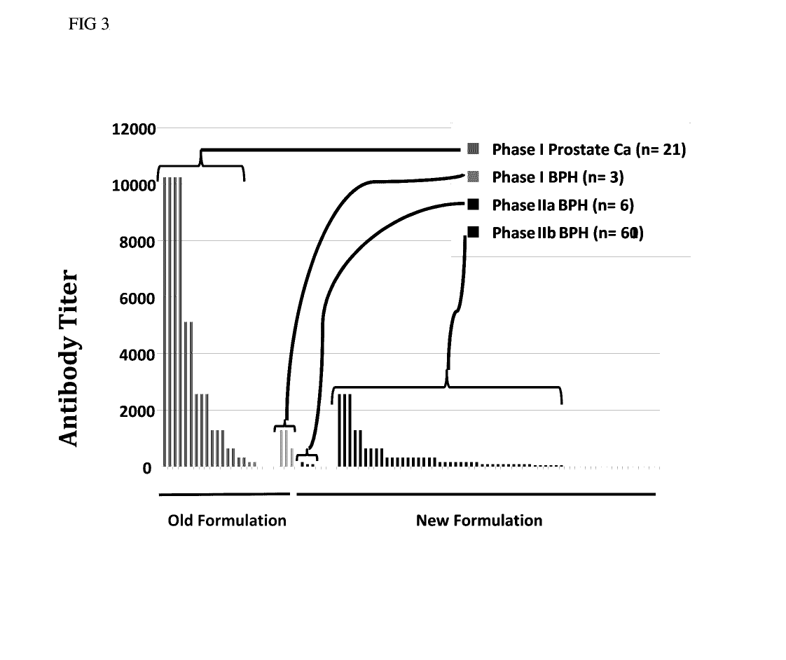

Antibody titers were determined Pre-Dose and at Days 30, 60 and 90. Analysis showed 21 of the 24 patients had samples at day 90 that could be assayed for antibody titers (FIG. 3). Of the 21 (day 90) samples, 18/21 (86%) had antibody titers of ≧160. Titers equal to or above 1:1280 were consistently determined to be able to neutralize the toxicity of PRX302.

The second human clinical trial with PRX302 was a Phase I dose escalation study is subjects requiring treatment for BPH. PRX302 was injected transperineally into the prostate. Subjects received total doses of PRX302 (which was administered according to the weight of the prostate of each subject) ranging from 1.13 to 21.0 μg in PBS-EDTA prepared as in the Phase I study above. A similar analysis to the one described above for the prostate cancer study was performed on 3 men in the Phase I BPH dose escalation study treated at the highest dose, which was approximately 21.0 μg. Day 90 antibody titers of 1/640 to 1/1280 were observed in all three men (100%) at this dose level (FIG. 3). As in the Phase I prostate cancer study, these antibodies were neutralizing with titers of 1:1280.

In summary, anti-PRX302 serum antibody neutralized the toxicity of PRX302 against this cell line. Therefore, PRX302-induced antibodies are neutralizing.

### Example 4

### Decreased Immunogenicity in the Presence of Albumin

In subsequent Phase IIa and Phase IIb BPH clinical trials, a new formulation of PRX302 was used. Initially this formulation was designed to eliminate the need for the step of coating the glass diluting vials with human serum albumin to prevent sticking as described in Example 2, which some practitioners found burdensome. Instead, HSA was added directly to the formulation of the therapeutic protein. This new formation included HSA at 2% (wt) and PRX302 in 20 mL of sterile PBS-EDTA which was administered intraprostatically to the subject. The presence of anti-PRX302 antibodies at various time points after administration of formulated PRS302 was determined as described in Example 2.

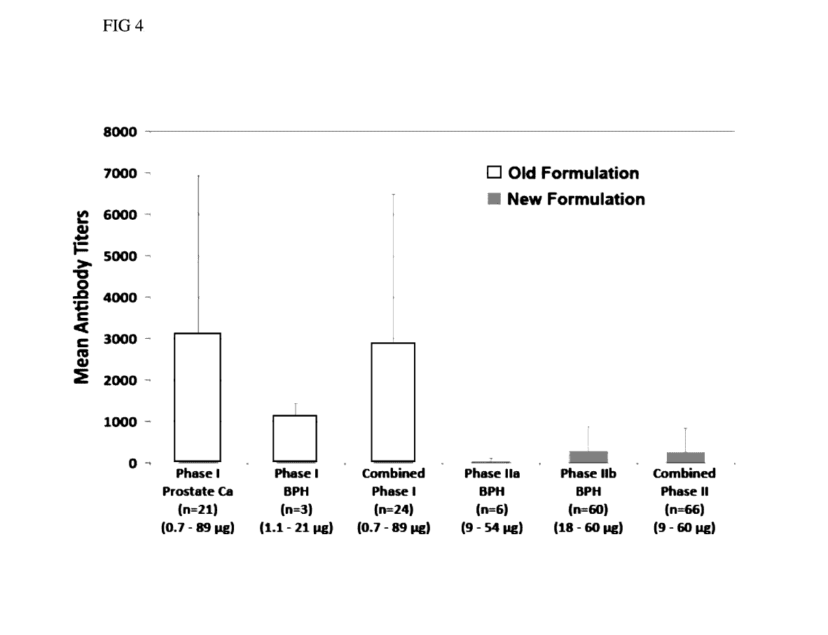

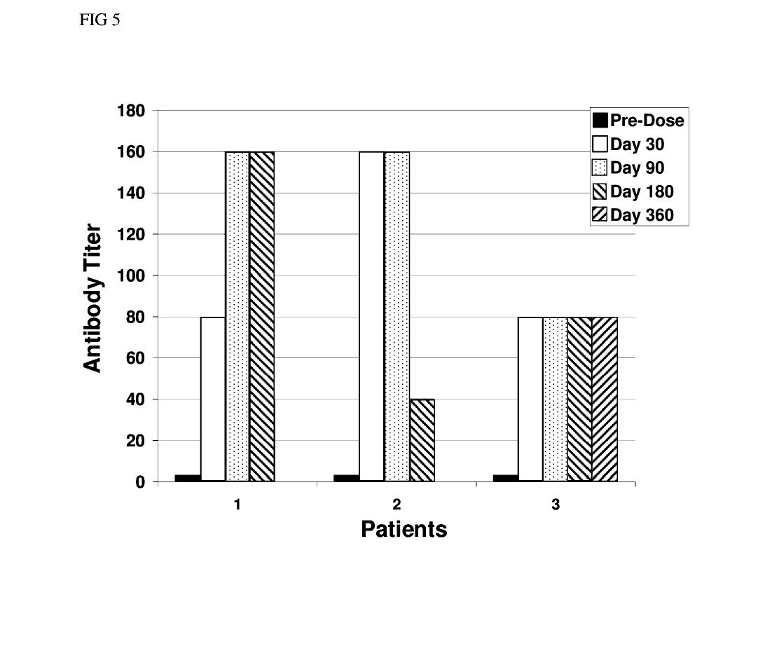

In the Phase IIa volume escalation study in men with moderate to severe BPH, 27.0-54.0 μg of PRX302 was injected into the prostate, depending on prostate size and cohort. The molar ratio of HSA:PRX302 in the formulations that were administered ranged from 2500:1 to 5000:1. In this study men were injected with higher total amounts of PRX302 compared to the highest dose cohort 4 of the phase I dose-escalation BPH study (15.75-21.0 μg). The total dose was also similar to levels injected in cohorts 5-7 of the Phase I study in recurrent localized prostate cancer patients (19.2-90.0 μg injected). In this Phase IIa study, antibody titers were measured in six men treated at highest dose cohort (cohort 3) at pre-dose and days 30, 90, 180 and 360. Day 90 titers were undetectable in 3/6 men and ≦160 in the remaining men. None of these 6 men had neutralizing antibody titers in this study (FIG. 4). Three men with measurable titers at day 90 had negative titers of antibody at day 360 (FIG. 5).

In the Phase IIb randomized double blind study in patients with moderate to severe BPH antibody titers are available at day 90 post PRX302 injection in 60 patients.

The ability of these antibodies to neutralize PRX302 can be determined as in Example 3.

In summary, 18/60 patients (30%) had no detectable antibodies at day 90 (FIG. 3). Earlier studies (Phase I prostate cancer and Phase I BPH) in which antibody titers were consistently neutralizing only at titers ≧1280, only 5/60 patients (8.3%) had titers in the Phase IIb randomized study that reached this level (FIG. 3). The differences in these antibody titers between the Phase I and Phase II studies appears to be due to the addition of HSA to the PRX302 formation. Combined data comparing the results from Phase I (old formulation) and Phase II studies (new formulation) indicates that 21/24 patients (87.5%) in the combined Phase I studies had detectable antibodies at day 90, whereas 49/60 (81.6%) had detectable antibodies in the combined phase II studies (FIG. 4). However, the average titer of antibody in the Phase I studies was 2840 while the average titer in the Phase II studies was ˜10-fold lower at 278 (FIG. 4). In addition, the neutralizing threshold of 1:1280 was observed in 14/24 (58%) patients on the combined Phase I studies at day 90. In contrast, this level of antibody was only observed in 5/66 (7.5%) of patients in the combined Phase II studies.

Subjects in a Phase II prostate cancer clinical trial were treated with PRX302 (dosage ranging from 102 μg to 480 μg, depending on the size of the prostate) as described in Example 2. The PRX302 was administered in a solution of 2% HSA ranging in volume from 3.4 ml to 9.6 ml (depending on the size of the prostate). The molar ratio of HSA to PRX302 in the formulation was approximately 525:1. Antibody titers were determined pre-dose and at Days 45, 90, 180, and 270. All subjects showed antibody titers at day 45 and 4 of 6 men had samples available for day 90 analysis. Of these 4 samples from day 90, all had antibody titers of ≧160 (i.e., titer above potential false positive level and ¼ (25%) subjects had titers above 1:1280. In summary, these results demonstrate that a single injection of PRX302 without albumin into the prostate can induce neutralizing titers of IgG class antibodies. In contrast, the new formulation of PRX302 for intraprostatic injections which includes HSA, on average reduced titers of antibodies by ˜10-fold as compared to the absence of HSA. In addition, neutralizing levels of antibody were only observed in 5/66 (7.5%) of patients treated on these two studies.

These data indicate that the new formulation of PRX302 which includes HSA will allow for repeat dosing if required. Thus, patients could be re-dosed at some point with negligible antibody titers. Data from long term evaluation of antibody titers performed in the Phase Ha BPH study indicate that titers become undetectable after one year post treatment with PRX302 (FIG. 5).

### Example 5

### Effect of HSA on Vial Adsorption

In the Phase 1 BPH study, a subject dosing pack contained one PRX302 drug product vial (3 mg/mL), 2 diluent vials, each containing 15 mL diluent (10 mM sodium phosphate, 150 mM sodium chloride and 1 mM ethylenediamine tetraacetic acid [EDTA]) and two 50 mL empty mixing vial. Each subject dosing pack was accompanied by 1 vial of 20% HSA solution. The dosing solution preparation procedure was carried out as follows: 4.5 mL of diluent from one of the diluent vials was transferred aseptically to one of the mixing vials followed by the addition of 0.5 mL of 20% HSA and mixed gently to obtain a 2% HSA solution. This 2% HSA solution was swirled several times in the mixing vial to ensure that the inner surface of the vial was in complete contact with the HSA solution and the contents poured-out and discarded appropriately. A similar coating process was used in the Phase 1 prostate cancer study.

A study was conducted which showed that this coating procedure significantly reduced adsorption of PRX302 to the inner surface of the mixing vial. After 4 hours approximately 60% of PRX302 remained in solution in the uncoated mixing vial whereas 95% and above of PRX302 remained in solution in the coated vial. Furthermore, after 24 hours, approximately 10% of PRX302 remained in solution in the uncoated mixing vial, whereas 46% of PRX302 remained in solution in the coated mixing vial.

### Example 6

### Effect of HSA on PRX302 Potency In Vitro

A potency assay was conducted to determine the in vitro biological activity of PRX302 dosing solution (3 μg/mL) with and without 2% HSA, as well as PRX302 in the 2% HSA-coated mixing vial. This assay used PSA-producing LNCaP human prostate cancer cells. LNCaP cells express moderate levels of PSA which are able to proteolytically cleave PRX302 into its active form thereby forming PRX302 heptamers that are able to insert into the membrane of the LNCaP cells, leading to cell death.

In order to compare the effect of these conditions, a PRX302 reference standard was used. This standard was created using 3 mg/mL PRX302 which was diluted in PBS/EDTA to 0.1 mg/mL and 0.003 mg/mL (3 μg/mL) reference solutions. These dilutions of PRX302 reference standard were performed in Eppendorf tubes in order to prevent adsorption.

This potency assay was performed in 3 different assay plates with 2 different lots of LNCaP cells. LNCaP cells were seeded in 3 assay plates at 10,000 cells per well in flat-bottom 96-well plates at a volume of 100 μL per well and incubated for 3 to 3.5 hours at 37° C. and 5% CO₂. Dilutions of PRX302 (PRX302 alone as a reference standard, PRX302 with 2% HSA from 2 different sources and PRX302 in mixing vial coated with 2% HSA) were prepared in duplicate in round-bottom 96-well plates. Fifty microliters (50 μL) from each well was added to the cell culture plates and incubated for 72 hours at 37° C. and 5% CO₂. After 72 hours of incubation, 30 μL of Promega CellTiter 96 Aqueous One Solution was added to each well and the plates were incubated for a further 120±10 minutes at 37° C. and 5% CO₂. Optical density was measured at 490 nm and the data was plotted as OD490 nm versus PRX302 concentration in GraphPad prism software. The median effective concentration (EC₅₀) value is defined as the concentration of PRX302 required to kill 50% of the cells.

Results from the above study show that HSA had no effect on the biological activity of PRX302. Biological activity, measured in term of potency (EC₅₀ value) of 3 μg/mL PRX302 dosing solution with and without 2% HSA along with PRX302 in the mixing vial coated with 2% HSA are comparable to each other as well as to the PRX302 reference standard.

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. I therefore claim as my invention all that comes within the scope and spirit of these claims.

1. A pharmaceutical composition, comprising:
albumin; and
a modified proaerolysin protein, wherein the modified proaerolysin protein comprises a prostate-specific protease cleavage site that replaces a proaerolysin furin cleavage site corresponding to amino acids 427-432 of SEQ ID NO: 2.

2. The composition of claim 1, wherein said albumin is human serum albumin.

3. The composition of claim 2, wherein the albumin is a recombinant human serum albumin or a human serum albumin purified from human blood.

4. The composition of claim 2, wherein said therapeutic protein is a modified proaerolysin protein selected from SEQ ID NO: 4 and SEQ ID NO: 28.

5. The composition of claim 4, wherein said therapeutic protein is the protein of SEQ ID NO: 4.

6. The composition of claim 4, wherein said therapeutic protein is the protein of SEQ ID NO: 28.

7. The composition of claim 6, wherein the molar ratio of the amount of human serum albumin to the amount of therapeutic protein is between 5:1 and 100,000:1.

8. The composition of claim 7, wherein the molar ratio of the amount of human serum albumin to the amount of therapeutic protein is between 50:1 and 5,000:1.

9. The composition of claim 6, wherein the amount of human serum albumin in the pharmaceutical composition is between 0.01 and 25% by weight.

10. The composition of claim 7, wherein the amount of human serum albumin in the pharmaceutical composition is between 0.2 and 5% by weight.

11. The composition of claim 8, wherein the amount of human serum albumin in the pharmaceutical composition is between 1.8 and 2.2% by weight.

12. The composition of claim 9, wherein the amount of human serum albumin in the pharmaceutical composition is about 2% by weight.

13. The composition of claim 6, wherein the amount of the protein of SEQ ID NO: 28 is between 2 and 4 μg/ml.

14. The composition of claim 13, wherein the amount of the protein of SEQ ID NO: 28 is between 2.5 and 3.5 μg/ml.

15. The composition of claim 14, wherein the amount of the protein of SEQ ID NO: 28 is between 2.8 and 3.2 μg/ml.

16. The composition of claim 15, wherein the amount of the protein of SEQ ID NO: 28 is about 3 μg/ml.

17. The composition of claim 16, wherein the amount of human serum albumin in the pharmaceutical composition is about 2% by weight.

18. The composition of claim 17, comprising an aqueous solution of:
between 0.05 and 25.0 μg/ml of the protein of SEQ ID NO: 28;
between 1.5 and 2.5% human serum albumin by weight;
between 100 and 200 mM NaCl;
between 0.1 and 1.0 mM NaH₂PO₄;
between 5 and 15 mM Na₂HPO₄; and
between 0.5 and 1.5 mM disodium ethylenediaminetetraacetic acid.

19. The composition of claim 18, comprising:
between 0.5 and 10.0 μg/ml of the protein of SEQ ID NO: 28;
between 1.8 and 2.2% human serum albumin by weight;
between 130 and 140 mM NaCl;
between 0.5 and 0.7 mM NaH₂PO₄;
between 7 and 9 mM Na₂HPO₄; and
between 0.8 and 1.0 mM disodium ethylenediaminetetraacetic acid.

20. The composition of claim 18, comprising:
between 0.5 and 10.0 μg/ml of the protein of SEQ ID NO: 28;
about 2% human serum albumin by weight;
about 138 mM NaCl;
about 0.6 mM NaH₂PO₄;
about 8.5 mM Na₂HPO₄; and
about 0.92 mM disodium ethylenediaminetetraacetic acid.

21. A kit, comprising:
a first container containing the therapeutic protein of SEQ ID NO: 28; and
a second container containing human serum albumin.

22. The kit of claim 21, wherein
the first container contains a solution comprising about 300 μg/ml of the therapeutic protein of SEQ ID NO: 28;
the second container contains a solution comprising about 2% human serum albumin; and
further comprising instructions to transfer the solution from the first container into the second container.

23. The kit of claim 22, wherein the second container contains a solution comprising:
between 0.5 and 10.0 μg/ml of the protein of SEQ ID NO: 28;
between 1.8 and 2.2% human serum albumin by weight;
between 130 and 140 mM NaCl;
between 0.5 and 0.7 mM NaH₂PO₄;
between 7 and 9 mM Na₂HPO₄; and
between 0.8 and 1.0 mM disodium ethylenediaminetetraacetic acid.

24. The kit of claim 23, wherein the second container contains a solution comprising:
between 0.5 and 10.0 μg/ml of the protein of SEQ ID NO: 28;
about 2% human serum albumin by weight;
about 138 mM NaCl;
about 0.6 mM NaH₂PO₄;
about 8.5 mM Na₂HPO₄; and
about 0.92 mM disodium ethylenediaminetetraacetic acid.

25. A method for treating benign prostatic hyperplasia in a subject comprising the administration of a therapeutically effective amount of the composition of claim 4 to a patient in need thereof.

26. The method of claim 25, wherein administration of the composition results in a reduction in prostate volume or weight or a reduction in the rate of prostate growth.

27. The method of claim 26, wherein the prostate size is reduced by at least 10%.

28. The method of claim 25, wherein the composition is administered intraprostatically.

29. The method of claim 28, wherein the amount of the protein of SEQ ID NO: 28 which exits from the prostate after administration is reduced in comparison with a composition which does not comprise human serum albumin.

30. The method of claim 29, wherein the amount of the protein of SEQ ID NO: 28 which exits from the prostate after administration is reduced by at least 1%, at least 2%, at least 5%, at least 10%, at least 15%, at least 20%, at least 30%, at least 40%, at least 50%, at least 60%, at least 70%, at least 80%, or at least 90%.

31. The method of claim 25, wherein the immune response is decreased in comparison with a composition which does not comprise human serum albumin.

32. The method of claim 31, wherein the immune response is an antibody response.

33. The method of claim 32, wherein the antibody response is decreased by at least 1%, at least 2%, at least 5%, at least 10%, at least 15%, at least 20%, at least 30%, at least 40%, at least 50%, at least 60%, at least 70%, at least 80%, or at least 90%.

34. The method of claim 33, wherein the antibody response is decreased by at least 90%.

35. The method of claim 33, wherein the antibody response is decreased 2-fold to 10-fold.

36. A method for treating prostate cancer in a subject comprising the administration of a therapeutically effective amount of the composition of claim 4 to a patient in need thereof.

37. The method of claim 36, wherein the subject has a localized prostate tumor.

38. The method of claim 36, wherein the subject has metastatic prostate cancer.

39. The method of claim 36, wherein administration of the composition results in a reduction in prostate tumor volume.

40. The method of claim 39, wherein the prostate tumor volume is reduced by at least 10%.

41. The method of claim 36, wherein the composition is administered intraprostatically.

42. The method of claim 41, wherein the amount of the protein of SEQ ID NO: 28 which flows out from the needle and prostate in conjunction with administration is reduced in comparison with a composition which does not comprise human serum albumin.

43. The method of claim 42, wherein the amount of the protein of SEQ ID NO: 28 which flows out from the needle and prostate in conjunction with administration is reduced by at least 1%, at least 2%, at least 5%, at least 10%, at least 15%, at least 20%, at least 30%, at least 40%, at least 50%, at least 60%, at least 70%, at least 80%, or at least 90%.

44. The method of claim 36, wherein the immune response is decreased in comparison with a composition which does not comprise human serum albumin.

45. The method of claim 44, wherein the immune response is an antibody response.

46. The method of claim 45, wherein the antibody response is decreased by at least 1%, at least 2%, at least 5%, at least 10%, at least 15%, at least 20%, at least 30%, at least 40%, at least 50%, at least 60%, at least 70%, at least 80%, or at least 90%.

47. The method of claim 46, wherein the antibody response is decreased by at least 90%.

48. The method of claim 45, wherein the antibody response is decreased 2-fold to 10-fold.

49. A method of decreasing an antibody response to a therapeutic protein of selected from SEQ ID NO: 4 and SEQ ID NO: 28 comprising administering the therapeutic protein to the subject in a pharmaceutical composition comprising a sufficient amount of albumin to decrease the subject's antibody response to the therapeutic protein.

50. The method of claim 49, wherein the antibody response is decreased by 10% to 90%.

51. The method of claim 50, wherein the antibody response is decreased by at least 90%.

52. The method of claim 49, wherein the antibody response is decreased 2-fold to 10-fold.

53. The method of claim 49, wherein the albumin is a human serum albumin.

54. The method of claim 53, wherein the albumin is a recombinant human serum albumin or a human serum albumin purified from human blood.

55. The method of claim 49, wherein the molar ratio of the amount of albumin to the amount of therapeutic protein is between 5:1 and 100,000:1 or between 50:1 and 5,000:1.

56. The method of claim 49, wherein the amount of albumin in the pharmaceutical composition is between 0.01 and 25% by weight, between 0.2 and 5% by weight, or 2% by weight.

57. A method of decreasing an antibody response to a therapeutic protein in a subject in need of treatment therewith, comprising administering the therapeutic protein to the subject in a pharmaceutical composition comprising a sufficient amount of albumin to decrease the subject's antibody response to the therapeutic protein.

58. The method of claim 57, wherein the antibody response is decreased by 10% to 90%.

59. The method of claim 58, wherein the antibody response is decreased by at least 90%.

60. The method of claim 57, wherein the antibody response is decreased 2-fold to 10-fold.

61. The method of claim 57, wherein the albumin is a human serum albumin.

62. The method of claim 61, wherein the albumin is a recombinant human serum albumin or a human serum albumin purified from human blood.

63. The method of claim 61, wherein the molar ratio of the amount of albumin to the amount of therapeutic protein is between 5:1 and 100,000:1 or between 50:1 and 5,000:1.

64. The method of claim 61, wherein the amount of albumin in the pharmaceutical composition is between 0.01 and 25% by weight, between 0.2 and 5% by weight, or 2% by weight.

65. The method of claim 61, wherein the therapeutic protein is a targeted cargo protein, wherein the targeted cargo protein comprises:
a targeting moiety that specifically binds to a target displayed by a target cell, and
a cargo moiety that exerts a biological effect on the target cell.

66. The method of claim 65, wherein the targeted cargo protein comprises one or more cargo moieties selected from aerolysin, proaerolysin, bouganin, abrin, ricin, Pseudomonas exotoxin, cholera toxin, diphtheria toxin, tetanus toxin, neural thread protein and Bad.

67. The method of claim 65, wherein the target is a receptor for IL-2, IL-4, IL-13, GM-CSF, EGF, proaerolysin toxin, diphtheria toxin, anthrax toxin and tetanus toxin.

68. The method of claim 65, wherein the targeting moiety comprises a ligand for a receptor for IL-2, IL-4, IL-13, GM-CSF, EGF, nicotinic acetylcholine, CD22 or GPI anchor protein.

69. The method of claim 65, wherein the targeted cargo protein comprises Pseudomonas exotoxin linked to circularly permuted IL-4, IL-2 linked to aerolysin, IL-2 linked to proaerolysin, IL4 linked to BAD, GMCSF linked to BAD, EGF linked to proaerolysin, anti-EpCAM antibody linked to Pseudomonas exotoxin, anti-EpCAM antibody linked to bouganin, anti-mesothelin antibody linked to Pseudomonas exotoxin, anti-CD22 antibody linked to Pseudomonas exotoxin, anti-CD22 antibody linked to RNase A, and anti-PSMA antibody linked to thapsigargin.

70. The method of claim 69, wherein the therapeutic protein comprises Pseudomonas exotoxin linked to circularly permuted IL-4 and the molar ratio of the amount of the therapeutic protein to the amount of albumin is 5:1 to 5000:1, such as 50:1.

71. The method of claim 61, wherein the therapeutic protein comprises an apoptosis-modifying fusion protein comprising an inactive toxin protein domain, an apoptosis regulating protein domain, wherein the inactive toxin protein domain targets the fusion protein to the cell and is not biologically active.

72. The method of claim 71, wherein the apoptosis regulating protein domain comprises a Bcl-2 protein.

73. The method of claim 72, wherein the Bcl-2 protein is a pro-apoptotic protein selected from Bcl-xs, Bax, Bad, Bak, DIVA, Bak, Bik, Bim, Bid and Egl-1, or an anti-apoptotic protein selected from Bcl-xL, Mcl-1, Ced-9 and A1.

74. The method of claim 72, wherein the inactive toxin protein domain comprises a domain derived from diphtheria toxin, tetanus toxin or anthrax toxin.

75. A pharmaceutical composition comprising:
albumin; and
a therapeutic protein selected from the group consisting of:
an apoptosis-modifying fusion protein comprising an inactive toxin protein domain and an apoptosis regulating protein domain, wherein the inactive toxin protein domain targets the fusion protein to the cell and is not biologically active, or
a targeted cargo protein, comprising a targeting moiety that specifically binds to a target displayed by a target cell, and a cargo moiety that exerts a biological effect on a target cell.

76. The composition of claim 75, wherein the albumin is human serum albumin.

77. The composition of claim 76, wherein the apoptosis regulating protein domain comprises a Bcl-2 protein domain.

78. The composition of claim 77, wherein the Bcl-2 protein is Bcl-X_{L}.

79. The composition of claim 78, wherein the Bcl-X_{L} comprises amino acid residues 1-209 of Bcl-X_{L}.

80. The composition of claim 76, wherein the inactive toxin protein domain comprises an inactive anthrax toxin domain or an inactive diphtheria toxin domain.

81. The composition of claim 80, wherein the inactive anthrax toxin domain comprises an amino terminal portion of mature anthrax lethal factor (LF).

82. The composition of claim 81, wherein the amino terminal portion comprises amino acid residues 1-255 of mature anthrax LF.

83. The composition of claim 76, wherein the therapeutic protein comprises at least 95% sequence identity to the amino acid sequence shown in SEQ ID NO: 26.

84. The composition of claim 80, wherein the inactive diphtheria toxin domain comprises the translocation region (domain or sub-domain) of diphtheria toxin.

85. The composition of claim 77, wherein the therapeutic protein comprises at least 95% sequence identity to the amino acid sequence shown in SEQ ID NO: 20 or 22.

86. A method for modifying apoptosis in a target cell, comprising contacting the target cell with an amount of the composition of claim 75, sufficient to inhibit apoptosis.

87. The method of claim 86, wherein apoptosis in the target cell is inhibited.

88. The method of claim 87, wherein the target cell is a neuron, a lymphocyte, a macrophage, an epithelial cell, or a stem cell.

89. The method of claim 86, wherein apoptosis in the target cell is enhanced.

90. The method of claim 89, further comprising the step of co-administering an agent selected from the group consisting of a chemotherapeutic agent, an anti-inflammatory agent, an anti-viral agent, and an antibiotic agent.

91. The protein of claim 89, wherein the target cell is a tumor cell, a cancer cell, a neoplasm cell, a hyper-proliferative cell, or an adipocyte.

92. A method of reducing apoptosis in a subject after transient ischemic neuronal injury, comprising administering to the subject a therapeutically effective amount of the composition of claim 75.

93. The method of claim 92, wherein the transient ischemic neuronal injury is a spinal cord injury.


SEQUENCE LISTING (28 sequences)

SEQ ID NO: 1 - DNA, 1410 bp, Aeromonas hydrophilia
  CDS (1)..(1410): wild-type proaerolysin cDNA
gcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctggatattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaacccaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaaccgtccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacagcaaggtgcgtcgtgctcgcagtgtggacggcgctggtcaaggcctgaggctggagatcccgctcgatgcgcaagagctctccgggcttggcttcaacaacgtcagcctcagcgtgacccctgctgccaatcaa

SEQ ID NO: 2 - protein, 470 aa, Aeromonas hydrophilia
AEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETQLSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSKVRRARSVDGAGQGLRLEIPLDAQELSGLGFNNVSLSVTPAANQ

SEQ ID NO: 3 - DNA, 1410 bp, Artificial Sequence
  Proaerolysin with a PSA sequence substituted for the furin site.
gcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctggatattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaacccaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaaccgtccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacagccattcctccaagctgcagagtgtggacggcgctggtcaaggcctgaggctggagatcccgctcgatgcgcaagagctctccgggcttggcttcaacaacgtcagcctcagcgtgacccctgctgccaatcaa

SEQ ID NO: 4 - protein, 470 aa, Artificial Sequence
  Proaerolysin with a PSA sequence substituted for the furin site.
AEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETELSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSHSSKLQSVDGAGQGLRLEIPLDAQELSGLGFNNVSLSVTPAANQ

SEQ ID NO: 5 - protein, 6 aa, Homo Sapiens
HSSKLQ

SEQ ID NO: 6 - protein, 8 aa, Artificial Sequence
  PSA cleavage site
HSSKLQSA

SEQ ID NO: 7 - protein, 6 aa, Artificial Sequence
  PSA cleavage site
QFYSSN

SEQ ID NO: 8 - protein, 7 aa, Artificial Sequence
  PSA cleavage site
GISSFQS

SEQ ID NO: 9 - protein, 7 aa, Homo sapiens
KGISSQY

SEQ ID NO: 10 - protein, 7 aa, Homo sapiens
SRKSQQY

SEQ ID NO: 11 - protein, 7 aa, Homo sapiens
ATKSKQH

SEQ ID NO: 12 - protein, 7 aa, Homo sapiens
KGLSSQC

SEQ ID NO: 13 - protein, 7 aa, Homo sapiens
LGGSSQL

SEQ ID NO: 14 - protein, 7 aa, Homo sapiens
EHSSKLQ

SEQ ID NO: 15 - protein, 4 aa, Homo sapiens
SKLQ

SEQ ID NO: 16 - protein, 10 aa, Homo sapiens
QHWSYGLRPG

SEQ ID NO: 17 - protein, 10 aa, Artificial Sequence
  LHRH variant sequence.
EHWSYKLRPG

SEQ ID NO: 18 - DNA, 1236 bp, Artificial Sequence
  Description of Artificial Sequence genetic fusion
atgggccatcatcatcatcatcatcatcatcatcacagcagcggccatatcgaaggtcgtatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaagcgtattctgcggccgcgcataaaacgcaaccatttcttcatgacgggtatgctgtcagttggaacactgttgaagattcgataatccgaactggttttcaaggggagagtgggcacgacataaaaattactgctgaaaataccccgcttccaatcgcgggtgtcctactaccgactattcctggaaagctggacgttaataagtccaagactcatatttccgtaaatggtcggaaaataaggatgcgttgcagagctatagacggtgatgtaactttttgtcgccctaaatctcctgtttatgttggtaatggtgtgcatgcgaatcttcacgtggcatttcacagaagcagctcggagaaaattcattctaatgaaatttcgtcggattccataggcgttcttgggtaccagaaaacagtagatcacaccaaggttaattctaagctatcgctattttttgaaatcaaaagctga

SEQ ID NO: 19 - protein, 411 aa, Artificial Sequence
  Description of Artificial Sequence genetic fusion
MGHHHHHHHHHHSSGHIEGRMSQSNRELVVDFLSYKLSQKGYSWSQFSDVEENRTEAPEGTESEMETPSAINGNPSWHLADSPAVNGATAHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRKAYSAAAHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 20 - DNA, 1704 bp, Artificial Sequence
  Description of Artificial Sequence genetic fusion
atgggccatcatcatcatcatcatcatcatcatcacagcagcggccatatcgaaggtcgtcatatgggaaccccaaagcagccctcgctggctcctgcacacgccctaggcttgaggaagtccgatcccggaatccggagcctggggagcgacgcgggaggaaggcggtggagaccagcagcccagagtatgttccagatcccagagtttgagccgagtgagcaggaagacgctagtgctacagataggggcctgggccctagcctcactgaggaccagccaggtccctacctggccccaggtctcctggggagcaacattcatcagcagggacgggcagccaccaacagtcatcatggaggcgcaggggctatggagactcggagtcgccacagtgcgtacccagcggggaccgaggaggatgaagggatggaggaggagcttagcccttttcgaggacgctcgcgtgcggctccccccaatctctgggcagcgcagcgctacggccgtgagctccgaaggatgagcgatgagtttgagggttccttcaagggacttcctcgcccaaagagcgcaggcactgcaacacagatgcgacaaagcgccggctggacgcgcattatccagtcctggtgggatcgaaacttgggcaaaggaggctccaccccctcccagtcagtaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctccggggcataaaacgcaaccatttcttcatgacgggtatgctgtcagttggaacactgttgaagattcgataatccgaactggttttcaaggggagagtgggcacgacataaaaattactgctgaaaataccccgcttccaatcgcgggtgtcctactaccgactattcctggaaagctggacgttaataagtccaagactcatatttccgtaaatggtcggaaaataaggatgcgttgcagagctatagacggtgatgtaactttttgtcgccctaaatctcctgtttatgttggtaatggtgtgcatgcgaatcttcacgtggcatttcacagaagcagctcggagaaaattcattctaatgaaatttcgtcggattccataggcgttcttgggtaccagaaaacagtagatcacaccaaggttaattctaagctatcgctattttttgaaatcaaaagctga

SEQ ID NO: 21 - protein, 567 aa, Artificial Sequence
  Description of Artificial Sequence genetic fusion
MGHHHHHHHHHHSSGHIEGRHMGTPKQPSLAPAHALGLRKSDPGIRSLGSDAGGRRWRPAAQSMFQIPEFEPSEQEDASATDRGLGPSLTEDQPGPYLAPGLLGSNIHQQGRAATNSHHGGAGAMETRSRHSAYPAGTEEDEGMEEELSPFRGRSRAAPPNLWAAQRYGRELRRMSDEFEGSFKGLPRPKSAGTATQMRQSAGWTRIIQSWWDRNLGKGGSTPSQSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 22 - DNA, 18 bp, Artificial Sequence
  Description of Artificial Sequence oligonucleotide linker
gcgtattctgcggccgcg

SEQ ID NO: 23 - protein, 6 aa, Artificial Sequence
  Description of Artificial Sequence oligopeptide linker
AYSAAA

SEQ ID NO: 24 - DNA, 1455 bp, Artificial Sequence
  Description of Artificial Sequence genetic fusion
atgggcagcagccatcatcatcatcatcacagcagcggcctggtgccgcgcggcagccatatggcgggcggtcatggtgatgtaggtatgcacgtaaaagagaaagagaaaaataaagatgagaataagagaaaagatgaagaacgaaataaaacacaggaagagcatttaaaggaaatcatgaaacacattgtaaaaatagaagtaaaaggggaggaagctgttaaaaaagaggcagcagaaaagctacttgagaaagtaccatctgatgttttagagatgtataaagcaattggaggaaagatatatattgtggatggtgatattacaaaacatatatctttagaagcattatctgaagataagaaaaaaataaaagacatttatgggaaagatgctttattacatgaacattatgtatatgcaaaagaaggatatgaacccgtacttgtaatccaatcttcggaagattatgtagaaaatactgaaaaggcactgaacgtttattatgaaataggtaagatattatcaagggatattttaagtaaaattaatcaaccatatcagaaatttttagatgtattaaataccattaaaaatgcatctgattcagatggacaagatcttttatttactaatcagcttaaggaacatcccacagacttttctgtagaattcttggaacaaaatagcaatgaggtacaagaagtatttgcgaaagcttttgcatattatatcgagccacagcatcgtgatgttttacagctttatgcaccggaagcttttaattacatggataaatttaacgaacaagaaataaatctatccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgc

SEQ ID NO: 25 - protein, 485 aa, Artificial Sequence
  Description of Artificial Sequence genetic fusion
MGSSHHHHHHSSGLVPRGSHMAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSMSQSNRELVVDFLSYKLSQKGYSWSQFSDVEENRTEAPEGTESEMETPSAINGNPSWHLADSPAVNGATAHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQER

SEQ ID NO: 26 - DNA, 1757 bp, Artificial Sequence
  IL-4 linked to Pseudomonas exotoxin
gcttgcatgcctgcagaagaaggagatatacatatgcaaaaaataaaactaactggcttgtcattaatcatatccggcctgctgatggcacaggcgcaagcggcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctggatattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctagatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaaccgaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaaccgtccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacagccattcctccaagctgcagagtgtggacggcgctggtcaaggcctgaggctggagatcccgctcgatgcgcaagagctctccgggcttggcttcaacaacgtcagcctcagcgtgacccctgctgccaatcaacatcatcatcatcatcattaacggcagcgctaacaacatcatcatcatcatcattaacggcagcgcgttgtagtgatggaaccgggcctctggcccggtttttgtttgcactggtcgggcttgttaaaggcttgtgctttccatttccccacttatactggcgccatcttgtcggagtgccaaccgtcgaacgacgcgaggctgagaccgttaattcgggatccgtggaacctgatccccgggaa

SEQ ID NO: 27 - protein, 1757 aa, Artificial Sequence
  IL-4 linked to Pseudomonas exotoxin
GCTTGCATGCCTGCAGAAGAAGGAGATATACATATGCAAAAAATAAAACTAACTGGCTTGTCATTAATCATATCCGGCCTGCTGATGGCACAGGCGCAAGCGGCAGAGCCCGTCTATCCAGACCAGCTTCGCTTGTTTTCATTGGGCCAAGGGGTCTGTGGCGACAAGTATCGCCCCGTCAATCGAGAAGAAGCCCAAAGCGTTAAAAGCAATATTGTCGGCATGATGGGGCAATGGCAAATAAGCGGGCTGGCCAACGGCTGGGTCATTATGGGGCCGGGTTATAACGGTGAAATAAAACCAGGGACAGCGTCCAATACCTGGTGTTATCCGACCAATCCTGTTACCGGTGAAATACCGACACTGTCTGCCCTGGATATTCCAGATGGTGACGAAGTCGATGTGCAGTGGCGACTGGTACATGACAGTGCGAATTTCATCAAACCAACCAGCTATCTGGCCCATTACCTCGGTTATGCCTGGGTGGGCGGCAATCACAGCCAATATGTCGGCGAAGACATGGATGTGACCCGTGATGGCGACGGCTGGGTGATCCGTGGCAACAATGACGGCGGCTGTGACGGCTATCGCTGTGGTGACAAGACGGCCATCAAGGTCAGCAACTTCGCCTATAACCTAGATCCCGACAGCTTCAAGCATGGCGATGTCACCCAGTCCGACCGCCAGCTGGTCAAGACTGTGGTGGGCTGGGCGGTCAACGACAGCGACACCCCCCAATCCGGCTATGACGTCACCCTGCGCTACGACACAGCCACCAACTGGTCCAAGACCAACACCTATGGCCTGAGCGAGAAGGTGACCACCAAGAACAAGTTCAAGTGGCCACTGGTGGGGGAAACCGAACTCTCCATCGAGATTGCTGCCAATCAGTCCTGGGCGTCCCAGAACGGGGGCTCGACCACCACCTCCCTGTCTCAGTCCGTGCGACCGACTGTGCCGGCCCGCTCCAAGATCCCGGTGAAGATAGAGCTCTACAAGGCCGACATCTCCTATCCCTATGAGTTCAAGGCCGATGTCAGCTATGACCTGACCCTGAGCGGCTTCCTGCGCTGGGGCGGCAACGCCTGGTATACCCACCCGGACAACCGTCCGAACTGGAACCACACCTTCGTCATAGGTCCGTACAAGGACAAGGCGAGCAGCATTCGGTACCAGTGGGACAAGCGTTACATCCCGGGTGAAGTGAAGTGGTGGGACTGGAACTGGACCATACAGCAGAACGGTCTGTCTACCATGCAGAACAACCTGGCCAGAGTGCTGCGCCCGGTGCGGGCGGGGATCACCGGTGATTTCAGTGCCGAGAGCCAGTTTGCCGGCAACATAGAGATCGGTGCTCCCGTGCCGCTCGCGGCTGACAGCCATTCCTCCAAGCTGCAGAGTGTGGACGGCGCTGGTCAAGGCCTGAGGCTGGAGATCCCGCTCGATGCGCAAGAGCTCTCCGGGCTTGGCTTCAACAACGTCAGCCTCAGCGTGACCCCTGCTGCCAATCAACATCATCATCATCATCATTAACGGCAGCGCTAACAACATCATCATCATCATCATTAACGGCAGCGCGTTGTAGTGATGGAACCGGGCCTCTGGCCCGGTTTTTGTTTGCACTGGTCGGGCTTGTTAAAGGCTTGTGCTTTCCATTTCCCCACTTATACTGGCGCCATCTTGTCGGAGTGCCAACCGTCGAACGACGCGAGGCTGAGACCGTTAATTCGGGATCCGTGGAACCTGATCCCCGGGAA

SEQ ID NO: 28 - protein, 476 aa, Artificial Sequence
  Proaerolysin with a PSA sequence substituted for the furin site and a polyhistidine tag
AEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETELSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSHSSKLQSVDGAGQGLRLEIPLDAQELSGLGFNNVSLSVTPAANQHHHHHH